(19) United States
(12) Patent Application Publication
(10) Pub. No.: US 2007/0026391 A1
(43) Pub. Date: Feb. 1, 2007

(54) Methods and compositions for identifying chemical or biological agents using multiplexed labeling and colocalization detection

(75) Inventors: Roland B. Stoughton, San Diego, CA (US); Michael R. Meyer, San Diego, CA (US); Daniel D. Shoemaker, San Diego, CA (US)

Correspondence Address: THE LAW OFFICE OF STEVEN G ROEDER, 5560 CHELSEA AVE, LA JOLLA, CA, 92037, US

(73) Assignee: GHC Technologies, Inc.

(21) Appl. No.: 11/398,998

(22) Filed: Apr. 6, 2006

Related U.S. Application Data
(60) Provisional application No. 60/670,552, filed on Apr. 11, 2005.
(60) Provisional application No. 60/670,553, filed on Apr. 11, 2005.

(51) Int. Cl. C12Q 1/70; C12Q 1/68

(52) U.S. Cl. 435/5; 435/6

(57) ABSTRACT

The present invention provides methods for detecting a target pathogenic agent, e.g., a virus, a bacterium, and/or a toxic substance, using colocalization detection. The invention also provides methods for parallel detection of different target pathogenic agents in a sample using multiplexed labeling and colocalization detection. The invention also provides kits comprising sets of probes for detecting pathogenic agents. The invention further provides computer systems and computer program products for carrying out the method of determining degrees of colocalization.

### RELATED APPLICATIONS

This Application claims the benefit on U.S. Provisional Application Ser. Nos. 60/670,552 filed on Apr. 11, 2005 and 60/670,553 filed on Apr. 11, 2005. The contents of U.S. Provisional Application Ser. Nos. 60/670,552 and 60/670,553 are incorporated herein by reference.

### 1. FIELD OF THE INVENTION

The present invention relates to methods for detecting a target chemical or biological agent, e.g., a virus, a bacterium, and/or a toxic substance, using colocalization detection. The invention also relates to methods for detection of different target chemical or biological agents in parallel in a sample using multiplexed labeling and colocalization detection.

### 2. BACKGROUND OF THE INVENTION

The increasing threat of emerging infectious diseases due to increases in travel among human populations and their encroachment on animal habitats (Zimmerman, B. E., and Zimmerman, D. J., 2003, Killer germs: microbes and diseases that threaten humanity, Rev. and updated ed., Contemporary Books, Chicago) and the growing threat from drug resistant pathogens have made vaccination, treatment, and diagnostics of pathogens more important. For example, nosocomial infections claim 75,000 lives a year, a rate that has continued to increase over the last 20 years (Andremont et al., 1996, Clin Microbiol Infect 1, 160-167; Jarvis, 2003, Infection 31 Suppl 2, 44-8; Clark, et al., 2003, Curr Opin Crit Care 9, 403-12). Diagnostic systems capable of rapid and sensitive detection of sepsis concomitant with identification of the causative agent and inference of its drug resistance properties in clinical and point-of-care situations are an indispensable part of any effective containment system.

Most of the infectious agents cannot be diagnosed reliably in clinical samples without lengthy culturing procedures. Many viruses have no clinically useful assays. There is an extremely limited capacity for a rapid diagnostic response to an epidemic outbreak event, and no way to determine reliably if a patient with symptoms consistent with exposure to a pathogen threat actually is infected with such a pathogen. Diagnostic information is crucial to support point of care allocations of medical countermeasures, quarantine decisions, and to identify covert outbreaks within the detect-to-protect window of opportunity. In many scenarios this information would make the difference between large loss of life and minimal loss of life.

There is a particularly pronounced shortfall between what current enabling technologies could provide in the way of DNA- and RNA-based infectious disease diagnosis and what is actually available. The speed and economy with which new and known pathogens can be sequenced, and the genomic sequence data already available, would support specific assays for nearly all known infectious agents. Genome-based methods ultimately have the greatest potential for accurate classification of threats. PCR-based assays are replacing culture-based methods and immunoassay methods as the gold standard for pathogen detection and identification. PCR assay sensitivities and specificities are being demonstrated in many cases to be superior to traditional diagnostic alternatives (Druce et al., 2005, J Med Virol 75, 122-9; Paule et al., 2004, J Mol Diagn 6, 191-6; Xu et al., 2004, Ann Clin Microbiol Antimicrob 3, 21). Diagnostics for host specific antigenic responses often will fail during the critical detect-to-protect window of therapeutic opportunity because specific antibodies have not yet been adequately generated in the host. PCR diagnostic platforms have been developed commercially (LightCycler by Roche, RealArt by Artas, COBAS Amplicor by Roche) and point-of-care versions are in development such as the Lab in a Tube (Liat) by IQuum.

PCR-based assays have modest multiplexing capabilities for identifying multiple agents in the same reaction (da Silva Filho et al., 2004, Pediatr Pulmonol 37, 537-47). Variants of multiplex PCR have been developed which test in parallel for a number of distinct strains within a pathogen clade using a variable region interior to conserved priming regions (Ambretti et al., 2004, Anal Biochem 332, 349-57; Kim et al., 2005, FEMS Immunology and Medical Microbiology). Highly parallel assays for multiple agents that could cause a particular clinical presentation, such as ‘flu-like’ symptoms, are an obvious need. DNA microarray-based approaches to parallel assays are in early stages of development for clinical applications. In these approaches highly parallel amplification of many genomic regions is followed by microarray hybridization readout.

Various single molecule detection methods have been developed for direct detection of a target without resorting to any amplification scheme. Eigen et al. disclosed a method for detecting single molecules based on fluorescence correlation spectroscopy (Eigen et al., Proc. Natl. Acad Sci. USA 91:5740-5747; PCT publication WO 94/16313; U.S. Pat. Nos. 5,807,677; 5,849,545; 6,200,818; and 6,498,017). The method replies on monitoring spatial-temporal correlations between fluctuating fluorescence signals. In the method, fluorescence signal from a sample volume smaller than the “territory” of a single target molecule is recorded in a time-resolved manner. The size of the territory is reciprocal to the concentration of molecules.

PCT Publication No. WO 98/10097 discloses a method and apparatus for detection of single molecules using two-color fluorescence detection. The method involves labeling of individual molecules with at least two fluorescent probes of different emission spectrum. Simultaneous detection of the two labels indicates the presence of the molecule. The velocity of the molecule is determined by measuring the time required for the molecules to travel a fixed distance between two laser beams. Comparison of the molecule's velocity with that of standard species permits determination of the molecular weight of the molecule, which may be present in a concentration as small as one femtomolar.

Other techniques for characterizing single macromolecules include a method described in U.S. Pat. No. 5,807,677 for direct identification of a specific target nucleic acid sequence having a low copy number in a test solution. This method involves the preparation of a reference solution of a mixture of different short oligonucleotides. Each oligonucleotide includes a sequence complementary to a section of the target sequence and is labeled with one or more fluorescent dye molecules. The reference solution is incubated with the test solution under conditions favorable to hybridization of the short oligonucleotides with the nucleic acid target. The target sequence is identified in the solution by detection of the nucleic acid strands to which one or more of the labeled oligonucleotides are hybridized. To amplify the fluorescence signal, a “cocktail” of different oligonucleotides are used which are capable of hybridizing with sequences adjacent to but not overlapping with the target sequence.

High-content screens allow monitoring multiple molecules and/or processes. For example, high-content screens can be performed with multiple fluorescence labels of different colors (Giuliano et al., 1995, Curr. Op. Cell Biol. 7:4; Giuliano et al., 1995, Ann. Rev. Biophys. Biomol. Struct. 24:405). In a high-content screen, both spatial and temporal dynamics of various cellular processes can be monitored (Farkas et al., 1993, Ann. Rev. Physiol. 55:785; Giuliano et al., 1990, In Optical Microscopy for Biology. B. Herman and K. Jacobson (eds.), pp. 543-557, Wiley-Liss, New York; Hahn et al., 1992, Nature 359:736; Waggoner et al., 1996, Hum. Pathol. 27:494). Single cell measurements can also be performed. Each cell can be treated as a “well” that has spatial and temporal information on the activities of the labeled constituents.

Pathak et al. (Pathak et al., 2001, J. Am. Chem. Soc. 123:4103-4104) discloses a method using multicolor quantum dot tagged oligonucleotide probes for detection of chromosome abnormalities or mutations using fluorescence in situ hybridization (FISH) procedures.

Single-molecule DNA analytical methods which involve elongation of DNA molecule include optical mapping (Schwartz et al., 1993, Science 262:110-113; Meng et al., 1995, Nature Genet. 9:432; Jing et al., Proc. Natl. Acad. Sci. USA 95:8046-8051) and fiber-fluorescence in situ hybridization (fiber-FISH) (Bensimon et al., Science 265:2096; Michalet et al., 1997, Science 277:1518). In optical mapping, DNA molecules are elongated in a fluid sample and fixed in the elongated conformation in a gel or on a surface. Restriction digestions are then performed on the elongated and fixed DNA molecules. Ordered restriction maps are then generated by determining the size of the restriction fragments. In fiber-FISH, DNA molecules are elongated and fixed on a surface by molecular combing. Hybridization with fluorescently labeled probe sequences allows determination of sequence landmarks on the DNA molecules. Both methods require fixation of elongated molecules so that molecular lengths and/or distances between markers can be measured.

Han et al. (Han et al., 2001, Nature Biotechnology 19:631-635) describes a method of multicolor optical coding for biological assays by embedding quantum dots of different emission spectrum into polymeric microbeads at precisely controlled ratios. By adjusting the ratios of different quantum dots, wavelength and intensity multiplexed labeling of the beads can be achieve.

U.S. Patent Application Publication No. 20030013091 describes a method of generating a diverse population of uniquely labeled probes. In the method, target specific nucleic acid probes each having a different specifier and a corresponding population of anti-genedigits each having a unique label are generated. Each specifier consists of a particular combination of genedigits linked together. The genedigits as attachment points for the anti-genedigits. Thus, each specifier can have a particular combination of unique labels attached to it. The specifier can be detected based on its particular combination of unique labels.

Multi-color labeling and image analysis methods have been developed for determining colocalization of different fluorescence labels (see, e.g., Manders et al., 1992, Journal of Cell Science 103, 857-862; Manders et al., 1993, Journal of Microscopy 169: 375-382). For example, Steensel et al. (Steensel et al., Journal of Cell Science 109:787-792) studied the spatial distribution of transcription factors of glucocorticoid receptor (GR) and mineralocorticoid receptor (MR) in nuclei of CAI neurons by dual labeling immunocytochemistry and confocal microscopy. The MR was detected with a rabbit polyclonal antibody, followed by a FITC labeled anti-rabbit antibody; the GR was detected with a mouse monoclonal antibody, followed by a TRITC-conjugated anti-mouse antibody. Colocalization of the two labels was evaluated by calculating a Pearson correlation coefficient. It was found that both receptors are concentrated in about one thousand clusters within the nucleus. Some clusters contain either mineralocorticoid receptors or glucocorticoid receptors, but a significant number of clusters contain both receptors.

Koyama-Honda et al. (Koyama-Honda et al., Biophys J BioFAST, published on Dec. 13, 2004 as doi:10.1529/biophysj.104.048967) discloses a method for simultaneous, dual-color, single fluorescent molecule colocalization imaging, to quantitatively detect the colocalization of two species of individual molecules. The report showed that two individual molecules labeled with GFP and Alexa633 respectively can be detected and colocalized to within 64-100 nm (68-90% detectability) in the membrane of cells.

U.S. Pat. No. 5,962,238 discloses a method and apparatus for analyzing a material within a container, such as blood within a capillary in a volumetric cytometry system provides for detecting the edges of the container, counting the cells within the container, characterizing the cells within the container, and evaluating channels of data which contain information relevant to more than one of the detectable characteristics of the cells. A scanner scans a container of material including certain cells. Sampling circuitry is coupled to the scanner to generate scanned images of the material in the container. Two or more scanned images are generated based on fluorescence data from dyes that have overlapping spectra. The two scanned images are processed using a linear regression analysis among corresponding pixels in the scanned images near certain cells to characterize relative contents of two fluorescing dyes in a target cell. Target cells are identified from the scanned images using processing resources which identify a peak sample within a neighborhood, and compare the amplitude of the peak with the amplitude of pixels on the perimeter of the neighborhood. Upon identifying a target cell in this manner, data from the plurality of scanned images corresponding to the identified cell are saved for further analysis.

U.S. Pat. No. 6,844,150 discloses a novel optical ruler based on ultrahigh-resolution colocalization of single fluorescent probes is described. Two unique families of fluorophores are used, namely energy-transfer fluorescent beads and semiconductor nanocrystal (NC) quantum dots, that can be excited by a single laser wavelength but emit at different wavelengths. A multicolor sample-scanning confocal microscope was constructed which allows one to image each fluorescent light emitter, free of chromatic aberrations, by scanning the sample with nanometer scale steps using a piezo-scanner. The resulting spots are accurately localized by fitting them to the known shape of the excitation point-spread-function of the microscope.

U.S. Patent Application Publication US20020028457 discloses assays that allow for the detection of a single copy of a target of interest. The target species is either directly or indirectly labeled with a quantum dot. The Patent Publication also discloses assays that are based on the colocalization of two or more differently colored quantum dots on a single target species. The Patent Publication discloses uses of the assays including detection of nucleic acids, polypeptides, small organic bioactive agents (e.g., drugs, agents of war, herbicides, pesticides, etc.) and organisms.

Discussion or citation of a reference herein shall not be construed as an admission that such reference is prior art to the present invention.

### 3. SUMMARY OF THE INVENTION

The invention provides a method for determining whether a sample comprises a target pathogenic agent, said method comprising (a) determining quantitatively a degree of colocalization of a plurality of different probes on a surface, wherein any one or more pathogenic agents and/or cellular constituents therefrom from said sample are fixed on said surface, by calculating a metric of colocalization between a plurality of detection channels each corresponding to one of said probes, wherein each said different probe specifically binds a different one of a plurality of recognition sites, and wherein said plurality of different recognition sites are colocalized in said target pathogenic agent or a cellular constituent of said target pathogenic agent; and (b) determining that said sample comprises said target pathogenic agent if said degree of colocalization of said plurality of different probes on said surface is higher than a predetermined threshold.

In one embodiment, said step (a) is carried out by a method comprising (i) contacting said surface with a probe composition comprising said plurality of different probes under conditions that specific binding of said probes to their respective recognition sites occurs; (ii) detecting said plurality of different probes on said surface; and (iii) determining said degree of colocalization.

In a specific embodiment, the invention provides a method for determining whether a sample comprises a target pathogenic agent, said method comprising (a) contacting a surface, wherein any one or more pathogenic agents and/or cellular constituents therefrom from said sample are fixed on said surface, with a probe composition comprising a plurality of different probes under conditions such that specific binding of said probes to their respective recognition sites occurs, wherein each said different probe specifically binds a different one of a plurality of recognition sites, wherein said plurality of different recognition sites are colocalized in said target pathogenic agent or said cellular constituent; (b) detecting said plurality of different probes on said surface; (c) determining quantitatively a degree of colocalization of said plurality of different probes on said surface by calculating a metric of colocalization between a plurality of detection channels each corresponding to one of said probes; and (d) determining that said sample comprises said target pathogenic agent if said degree of colocalization of said plurality of different probes on said surface is higher than a predetermined threshold.

In the methods of the invention, said plurality of probes comprises 2, 3, 4, 5, or 6 different probes.

Each said different probe can be labeled with a different fluorescence label having a different emission and/or excitation wavelength.

Each of said different probe can also be labeled with a fluorescence label such that the plurality of different probes are labeled with a predetermined number of each of a plurality of different fluorescence labels. In one embodiment, said plurality of different probes is labeled with 2, 3, 4, or 5 different fluorescence labels. In one embodiment, each different fluorescence label has a different emission and/or excitation wavelength.

In another embodiment, at least one fluorescence label has a different excitation wavelength.

In a preferred embodiment, said plurality of recognition sites comprises a plurality of DNA sequences of said target pathogenic agent, wherein said DNA sequences are located in a 2 kb or less or 1 kb or less region of DNA sequence of said target pathogenic agent.

In another embodiment, said probe composition further comprising a type-specific label, e.g., DAPI, and said method further comprising detecting said type-specific label and determining colocalization of plurality of probes on image regions also labeled with said type-specific label.

In another preferred embodiment, said plurality of recognition sites comprises a plurality of surface antigens of said target pathogenic agent.

In one embodiment, said degree of colocalization is represented by a metric comprising an overlap coefficient of a pair of said plurality of detection channels.

In another embodiment, said degree of colocalization is represented by a metric comprising colocalization coefficients m₁ and m₂ of a pair of said plurality of detection channels.

In still another embodiment, said degree of colocalization is represented by a metric comprising at least a Pearson correlation coefficient of a pair of said plurality of detection channels.

In another embodiment, said target pathogenic agent further comprises a second plurality of different recognition sites that are colocalized, wherein said probe composition further comprises a second plurality of different probes each specifically binding one of said second plurality of recognition sites, wherein said method further comprises before step (d) repeating steps (b) and (c) with said second plurality of probes, and determining that said sample comprises said target pathogenic agent if a degree of colocalization of said second plurality of different probes on said surface is also higher than a second predetermined threshold. In one embodiment, said plurality of recognition sites comprises a plurality of DNA sequences of said target pathogenic agent, wherein said DNA sequences are located in a 1 kb or less region of DNA sequence of said target pathogenic agent, and wherein said second plurality of recognition sites comprises a plurality of surface antigens of said target pathogenic agent.

The invention also provides a method for determining whether a sample comprises a plurality of different target pathogenic agents, wherein each said target pathogenic agent comprises a plurality of different recognition sites that are colocalized, said method comprising (a) contacting a surface, wherein any one or more pathogenic agents and/or cellular constituents therefrom from said sample are fixed on said surface, with a probe composition comprising a plurality of sets of different probes under conditions that specific binding of said probes to their respective recognition sites occurs, wherein each said set comprises a plurality of different probes each specifically binding one of said plurality of recognition sites; (b) detecting said plurality sets of different probes on said surface; (c) determining quantitatively for each said set a degree of colocalization of said plurality of different probes on said surface by calculating a metric of colocalization between a plurality of detection channels each corresponding to one of said probes; and (d) determining that said sample comprises a target pathogenic agent if said degree of colocalization of the corresponding set of probes on said surface is higher than a predetermined threshold.

In preferred embodiments, said plurality of different target pathogenic agents comprises 5, 10, 25, 50, or 100 different target pathogenic agents.

In a specific embodiment, each of said sets of different probes comprises 3 different probes.

In one embodiment, each said different probe is labeled with one of ten different labels such that each set of different probes has a unique combination of different labels. In a specific embodiment, said ten different labels are ZnS-capped CdSe quantum dots having emission wavelengths at approximately 443, 473, 481, 500, 518, 543, 565, 587, 610, and 655 nm, respectively.

In another embodiment, said plurality of recognition sites comprises a plurality of DNA sequences of said target pathogenic agent, wherein said DNA sequences are located in a 2 kb or less or 1 kb or less region of DNA sequence of said target pathogenic agent.

In one embodiment, said probe composition further comprises a type-specific label, e.g., DAPI, and said method further comprises detecting said type-specific label and determining colocalization of plurality of probes on image regions also labeled with said type-specific label.

In one embodiment, said degree of colocalization is represented by a metric comprising an overlap coefficient of a pair of said plurality of detection channels.

In another embodiment, said degree of colocalization is represented by a metric comprising colocalization coefficients m₁ and m₂ of a pair of said plurality of detection channels.

In still another embodiment, said degree of colocalization is represented by a metric comprising at least a Pearson correlation coefficient of a pair of said plurality of detection channels.

In another embodiment, said predetermined threshold is determined using one or more reference samples, each comprising a predetermined number of copies of each said target pathogenic agent.

In a specific embodiment, the invention provides a method for determining whether a sample comprises a target nucleic acid or protein, said method comprising (a) determining quantitatively a degree of colocalization of a plurality of different probes on a surface, wherein any one or more nucleic acids or proteins from said sample are fixed on said surface, by calculating a metric of colocalization between a plurality of detection channels each corresponding to one of said probes, wherein each said different probe specifically binds a different one of a plurality of recognition sites, and wherein said plurality of different recognition sites are colocalized in said target nucleic acid or protein; and (b) determining that said sample comprises said target nucleic acid or protein if said degree of colocalization of said plurality of different probes on said surface is higher than a predetermined threshold. In one embodiment, said step (a) is carried out by a method comprising (i) contacting said surface with a probe composition comprising said plurality of different probes under conditions that specific binding of said probes to their respective recognition sites occurs; (ii) detecting said plurality of different probes on said, surface; and (iii) determining said degree of colocalization.

In another specific embodiment, the invention provides a method for determining whether a sample comprises a target nucleic acid or protein, said method comprising (a) contacting a surface, wherein any one or more nucleic acids or proteins from said sample are fixed on said surface, nucleic acids or proteins from said sample fixed on said surface with a probe composition comprising a plurality of different probes under conditions such that specific binding of said probes to their respective recognition sites occurs, wherein each said different probe specifically binds a different one of a plurality of recognition sites, wherein said plurality of different recognition sites are colocalized in said target nucleic acid or protein; (b) detecting said plurality of different probes on said surface; (c) determining quantitatively a degree of colocalization of said plurality of different probes on said surface by calculating a metric of colocalization between a plurality of detection channels each corresponding to one of said probes; and (d) determining that said sample comprises said target nucleic acid or protein if said degree of colocalization of said plurality of different probes on said surface is higher than a predetermined threshold.

The invention also provides a computer system comprising a processor and a memory coupled to said processor and encoding one or more programs, wherein said one or more programs cause the processor to carry out any one of the method of invention.

The invention also provides a computer program product for use in conjunction with a computer having a processor and a memory connected to the processor, said computer program product comprising a computer readable storage medium having a computer program mechanism encoded thereon, wherein said computer program mechanism may be loaded into the memory of said computer and cause said computer to carry out any one of the method of invention.

The invention also provides a kit comprising (a) in one or more containers a probe composition comprising for each of one or more pathogenic agents a set of two or more probes each specifically binding to a recognition site of said pathogenic agent; and (b) threshold value data on an accessible medium, e.g., printed on a data sheet or encoded on a computer readable medium, comprising colocalization threshold values for each of said one or more pathogenic agents, wherein said colocalization threshold values for each said pathogenic agent correspond to a degree of colocalization of said two or more probes in said set which indicates the presence or absence of said pathogenic agent.

In one embodiment, each of said sets of different probes in the kit comprises 3 different probes. In another embodiment, each said different probe is labeled with one of ten different labels such that each set of different probes has a unique combination of different labels. In a specific embodiment, said ten different labels are ZnS-capped CdSe quantum dots having emission wavelengths at approximately 443, 473, 481, 500, 518, 543, 565, 587, 610, and 655 nm, respectively.

In a preferred embodiment, said plurality of recognition sites comprises a plurality of DNA sequences of said target pathogenic agent, wherein said DNA sequences are located in a 2 kb or less or 1 kb or less region of DNA sequence of said target pathogenic agent.

In another embodiment, said probe composition further comprising a type-specific label, e.g., DAPI.

In preferred embodiments, said one or more pathogenic agents comprises 5, 10, 25, 50 or 100 different pathogenic agents.

In another embodiment, the set of probes for each said one or more pathogenic agents is in a separate container, and the kit further comprises reagents for constructing a probe composition using a portion or all of said sets of probes.

In preferred embodiments of the methods of the invention, said sample and/or cellular constituents therefrom has not been subject to in vitro amplification of nucleic acids, e.g., PCR amplication, prior to said obtaining step.

### 4. BRIEF DESCRIPTION OF FIGURES

FIG. 1 illustrates the process of the diagnostic method.

FIG. 2 Two-component receptor binding model was used to simulate kinetics of signal and clutter. Approach to equilibrium is shown at left, and result of stringent wash starting at t=100 sec is shown at right. Clutter comes from non-specific binding events at secondary binding sites that are assumed to be 10⁶ times more numerous than recognition sites, but have dissociation constant K=10⁻⁴ while recognition sites have K=10⁻¹⁰. An ‘on rate’ of 10⁵ s⁻¹ has been assumed. Multiple curves are for different applied ligand (antibody) concentrations from 1 μM down to 100 pM. Red dashed curves give number of non-specific binding events; blue solid curves give number of specific events. Vertical axis is scaled assuming 1000 copies of a molecular recognition site. The wash behavior assumed a five times faster dissociation for the specific binding. The dissociation rate for both specific and non-specific binding can be adjusted with stringency, so the time axis scale after 100 sec in the right hand plot should be interpreted as being arbitrary.

FIG. 3 Baculovirus virions on filter are detected with 60 sec incubation time and 10 sec wash. Left—gp64 antibody with fluorescent labeling via secondary antibody. Right—mismatched antibody for negative control. There were ˜10 virions per 10 μ² image pixel averaged over the filter region.

FIG. 4 Left—“green” channel showing 605 nm emission quantum dot labeled antibodies binding to E. coli. Right—both red and green channels showing both 605 nm and 705 nm labeled antibodies collocating on the E. coli.

FIG. 5 E. coli (round) and B. cereus (rod) cells are stained blue by DAPI for double stranded DNA (left). 605 nm emission quantum dot labeled polyclonal antibody to E. coli specifically stains the E. coli cells (right).

FIG. 6 Individual DNA fragments each labeled with one quantum dot are clearly detected with a one-second exposure. Field of view is ˜100 microns wide. Fragments appear as unresolved points. Negative controls confirmed that the signals were not from free dots left over from the dot-DNA conjugation and purification via gel electrophoresis.

FIG. 7 Gel-based separation of free quantum dots from dot-labeled DNA. DNA is dyed with SYBR green. Lane (1) DNA length markers. Lane (2) Free Streptavidin conjugated Qdots. Lane (3) Conjugation with 1 kb PCR product (no biotin). Lane (4) Conjugation with 1 kb PCR product (with biotin on both 5′ ends). Comparing Lanes 3 and 4 shows that the DNA mobility is decreased by the added Qdots. The sample imaged in FIG. 6 was taken from the lower orange band in Lane 4.

FIG. 8 Hybridized structures involving Qdot-labeled 50-mer oligos and 1-kb PCR products. Blue signal comes from SYBR green staining of double stranded DNA. “Green” and “red” signals come from 605 nm and 705 nm emission Qdot-labeled oligos. Field of view is about 40 μ.

FIG. 9 Two-minute hybridization of DNA Cy5-labeled probes to DNA target in solution, followed by 30 sec wash and imaging of bound probes on 2-mm wide filter, as in the final steps of FIG. 1. Right hand image is a negative control with probes only (no target DNA).

FIG. 10 Probe design for Ebola Zaire. Alignment of a region of the envelope glycoprotein gene is performed using known strains (rows). Regions of conservation are identified via an entropy measure (upper curve), and regions with likely cross-hybridization to known interfering organisms expected in the same sample are avoided. These criteria lead to the selected region whose alignment is displayed. In this case two different probe sequences are required to achieve adequate and homogenous binding energy (measured as melting temperature and displayed at right) over all the strains. The complement of the longer sequence will work for the lower 8 strains, and will have the melting temperature indicated with the gray bars at right. The shorter probe will work for the upper strains, with a melting temperature as shown by the dark bars at right.

FIG. 11 Assay cartridge operation.

FIG. 12 Instrument platform.

FIG. 13 Left panel: Individual DNA 50-mer probes each labeled with either a 605 nm emission ‘green’ or 705 nm emission ‘red’ quantum dot are clearly detected. Right panel: The two probe types were allowed to hybridize to their target recognition sites on a 1-kb DNA fragment. When both recognition sites receive probes, the target fragment emits both colors and appears ‘yellow’ (arrow).

FIGS. 14A-C illustrate quantitative colocalization determination of a 256×256 pixel image region containing E. coli cells after a 2-minute hybridization to Qdot-labeled antibodies of two different colors (605 nm “green”, 705 nm “red”). 14A: original image with intensity transform ‘gamma’ chosen to reveal background clutter associated with the individual labeled antibodies, as well as the bacterial cells. 14B: image composed of the pixel by pixel intensity product, illustrating improved signal-to-clutter ratio. 15C: intensity profile along the blue dashed line of FIG. 14B. The thick line is the product intensity, which has a much higher ratio of signal to noise across the bacteria features than do the individual color channels.

FIG. 15 Two different antibodies to Baculovirus gp64 surface protein were labeled with different quantum dot labels. Incubation and wash were accomplished via the methods of the invention in 5 minutes and 1 minute, respectively. The probe concentration was 40 nM. The average product of intensities between the two colors at different positions (x, x+D) was computed via digital Fourier Transform correlation of the microscope image, and the resulting circularly symmetric correlation function was averaged over position angle to yield a function of distance only (graph). A control experiment with no target virus (right panel) yielded little increase at small lags (lower curve in graph), whereas with the target present (left panel) a sharp increase at small lags corresponding to the 1-5 micron particle sizes is apparent in the correlation function (upper curve in graph).

### 5. DETAILED DESCRIPTION OF THE INVENTION

The present invention provides a method for determining whether a sample comprises a target chemical or biological agent, such as a pathogenic agent, e.g., a virus, a bacterium, a prion, or a toxic substance, or any other macromolecules, e.g., a DNA or a protein, using label multiplexing and colocalization detection. The sample can be any sample for which the existence of a target chemical or biological agent is to be determined.

In some embodiments, the sample is from an animal, e.g., a human or a non-human mammal, e.g., horses, cows, pigs, dogs, cats, sheep, goats, mice, rats, etc. The sample can be a body fluid, e.g., blood, urine, sputum, stool, and nasal swabs, or a tissue, e.g., swabs from areas of localized infection, e.g., skin and soft tissue. In other embodiments, the sample is from an environmental source, e.g., air, soil, or water. Thus, the method of the invention can be used for detecting infectious or toxic agents in a subject or in the environment.

The invention is particular useful for detecting a pathogenic agent. As used herein, a pathogenic agent refers to an agent that can cause a disease or any other undesirable conditions in an organism such as an animal, e.g., a human or a non-human mammal, or a plant. A pathogenic agent can be an infectious microorganism, e.g., a bacterium, a virus, or a prion. A pathogenic agent can be a toxin, or a pathogenic small molecule or macromolecule. A pathogen or toxin is also referred to as a “threat” in the application. The pathogenic agent preferably comprises a plurality of recognition sites that can be recognized by different probes. At least some of the recognition sites are preferably spatially located proximately with each other, i.e., colocalized, in the pathogenic agent or cellular constituents therefrom, e.g., nucleic acids and proteins. The recognition sites can be but are not limited to nucleic acid sequences, e.g., sequences in the genomic DNA, and proteins, e.g., surface antigens. In the application, pathogenic agents are often used as exemplary pathogenic agents to illustrate the methods of the invention. A person skilled in the art will understand that the methods of the invention are also applicable to other kinds of chemical or biological agents. In the application, the word “about” is often used to indicate approximation. For example, the term “about 1 minute” refers to a time period of approximately 1 minute.

The method of the invention involves contacting a sample with a plurality of different probes, e.g., different fluorescence labels that have different emission or excitation wavelength, which are specific to colocalized different recognition sites in the sample. If the sample comprises the target pathogenic agent, the probes bind their respective recognition sites. The labeled sample is then interrogated, e.g., by fluorescence imaging, to detect the plurality of different labels. The degree of colocalization of detected labels can then be determined, which provides an indication regarding whether the target pathogenic agent exists in the sample.

As used herein, colocalization refers to the presence of two or more recognition sites or probes, respectively, on an individual pathogenic agent or cellular constituent, e.g., nucleic acid or protein. In some embodiment, two or more molecular moieties are present at the same or proximate physical locations that their spatial separation cannot be resolved with the detection method used. For example, two or more nucleotide sequences located within a short distance, e.g., a few tens of bases, to each other along a DNA molecule may not be resolved spatially using conventional microscopy imaging. Other examples include different epitopes on the same protein, different eptitopes on each component of a protein complex, and so on. When these molecular moieties are labeled with distinguishable labels, colocalization of the labels are observed. For example, colocalization of two or more different fluorescence labels manifests as two or more spatially overlapped fluorescence wavelengths. In other embodiment, spatial separation may be resolved. Examples include surface antigens located on a bacterial cell at a distance greater than the spatial resolution of imaging method and nucleotide sequences in a nucleic acid which are separated by a distance greater than than the spatial resolution of imaging method.

As used herein, measurement of each different label is also referred to as a detection channel. For example, in fluorescence detection, each channel corresponds to one label having a particular emission or excitation wavelength. Thus, images consist of green and red fluorescence labels are referred to as having a green channel comprising measurements of the green label and a red channel comprising measurements of the red label. In some embodiments, different channels are acquired as different images. Colocalization of two different labels, e.g., green and red, is also referred to as colocalization of two channels.

The inventors have discovered that a target pathogenic agent having multiple recognition sites can be detected using multiple labels and detecting colocalization of the labels. The degree of colocalization of the multiple labels in the images or region(s) of the images provides a convenient, sensitive and accurate measure for determining the presence or absence of the pathogenic agent.

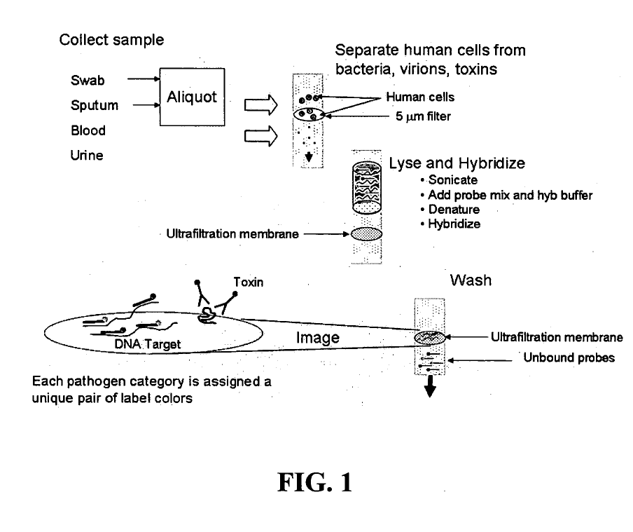

Thus, the method of the invention can be used for direct detection of unamplified target DNA and/or protein, e.g., genomic DNA or cellular mRNA without PCR amplication. Unique recognition sites for each pathogen are determined from bioinformatic analysis. Multiple recognition sites are chosen for each pathogen to assure robust detection and identification. The detection process is illustrated in FIG. 1. As shown in the lower left corner of the Figure, labels with different fluorescent colors are assigned to the multiple recognition probes; coincident detection of two or more colors assigned to a particular pathogen, for example, greatly increases the detection specificity.

### 5.1. Methods of Analyzing Biological Samples

The method of the invention utilizes multiple labels and colocalization detection to detect a pathogenic agent in a sample. Accurate colocalization determination in fluorescence microscopy can be achieved if emission spectra of the fluorochromes are sufficiently separated. To achieve this aim, fluorescence labels can be selected such that their emission wavelengths are sufficiently separated and can be resolved by the imaging device used. Depending on the spectral resolution of the detection device used, a person skilled in the art will be able to choose the appropriate labels that allow accurate colocalization determination. Conversely, if a particular set of labels is to be used, a person skilled in the art will be able to select the appropriate imaging device such that the labels can be separated and determined.

### 5.1.1. Label Coding and Multiplexing

The method of the invention employs different, distinguishable labels to achieve colocalization detection of a pathogenic agent. In one embodiment, a set of fluorescence labels having distinguishable emission wavelengths are used for labeling a pathogenic agent and/or cellular constituents therefrom. The set of fluorescence labels can consist of 2, 3, 4, 5, 6, 7, 8, 9, 10 or more different labels. The pathogenic agent is thus detected by detection of colocalization of the set of different fluorescence wavelengths. Such a labeling scheme is also referred to as “wavelength coding” or “color coding.” For example, a two-color encoding scheme can be achieved by using a green and a red label. In another embodiment, color encoding is combined with intensity encoding in which, in addition to utilizing a set of different labels, the relative number of each labels can be varied. For example, in the two-color encoding scheme using green and red, this can be achieved by varying the number of either the green or red or both, e.g., green:red 2:1, green:red 1:3, and so on. One advantage of using such combined color and intensity encoding is to increase the detection accuracy. Another advantage of using such combined color and intensity encoding is to increase the capacity of label multiplexing.

Direct detection of multiple markers on or within an intact pathogen can also be used to detect the pathogen in a sample. This can be achieved by monitoring multiple molecules and/or processes in the pathogen and/or a host cell without disrupting the cell. For example, multiple fluorescence labels of different colors can be to label different molecular species in a cell (Giuliano et al., 1995, Curr. Op. Cell Biol. 7:4; Giuliano et al., 1995, Ann. Rev. Biophys. Biomol. Struct. 24:405). The collection of measurements can include but not limited to a gene or gene transcript, a protein, a small cellular molecule, e.g., a metabolite, a measure of interactions between molecules, e.g., binding of a molecule to a protein, a measure of a molecular event/process, etc.

A pathogenic agent is determined to be present in the sample if the set of one or more probes that bind the pathogenic agent is detected in the appropriate detection channels.

To achieve efficient concurrent detection of a plurality of different pathogenic agents in a sample, label multiplexing can be used. In label multiplexing, a set of different labels is used to label all different pathogenic agents, each with a unique combination. In one embodiment of the invention, different pathogenic agents in the sample are probed by a set of different probes, each bound a different recognition site. The set of probes for each pathogenic agent is labeled with a unique combination of labels. Thus, each set of probes are detected as colocalization of the corresponding combination of labels. In one embodiment, m different probes each labeled with a different label from among a total of M distinguishable labels is used to uniquely label one pathogenic agent. The total multiplexing capacity of such a wavelength multiplexing embodiment can be determined according to equation

$$X = \frac{M !}{( M - m ) ! m !} \qquad ( 1 )$$

In another embodiment, m different probes each labeled with a same or different label from among a total of M distinguishable labels is used to uniquely label one pathogenic agent. The total multiplexing capacity of such a wavelength and intensity multiplexing embodiment can be determined according to equation

$$X = \frac{{M}^{m}}{m !} \qquad ( 2 )$$

In one embodiment, a set of fluorescent nanoparticles or quantum dots (QDs), e.g., semiconductor QDs such as ZnS-capped CdSe nanocrystals, are used as labels (see, e.g., Han et al., 2001, Nature Biotechnology 19:631-635; Pathak et al., 2001, J. Am. Chem. Soc. 123:4103-4104). The emission wavelengths of fluorescent QDs can be tuned by varying the sizes of the particle. For example, ZnS-capped CdSe QDs having ten distinguishable emission wavelengths at approximately 443, 473, 481, 500, 518, 543, 565, 587, 610, and 655 nm, respectively, can be used as labels. Comparing to organic fluorescence dyes, quantum dots offer higher brightness, narrower emission spectrum, higher bleaching stability, and single excitation source. In one embodiment, for each of a plurality of target pathogenic agents in a sample, a plurality of different probes each labeled with one different QD is used. In a specific embodiment, 2 different probes each labeled with a different QD from among a total of 10 distinguishable QDs is used to uniquely label one pathogenic agent, allowing detection of 45 different pathogenic agents. In a specific embodiment, 3 different probes each labeled with a different QD from among a total of 10 distinguishable QDs is used to uniquely label one pathogenic agent, allowing detection of 120 different pathogenic agents. In an embodiment using wavelength and intensity multiplexing, 4 different probes each labeled with a same or different QD from among a total of 10 distinguishable QDs is used to uniquely label one pathogenic agent, allowing detection of 10,000 different pathogenic agents.

In one embodiment, a type of biological molecules is also labeled with a label different from any of the labels that recognize specific recognition sites (“site-specific”). For example, DNA molecules can be labeled by a type of fluorescence dye molecules such as DPAI, or a DNA intercalator, such as YOYO. Such labels are referred to as labels specific for a particular type of pathogenic agents (“type-specific”). For example, DAPI and YOYO are DNA-specific labels. Overlapping fluorescence of type-specific and the appropriate site-specific labels can be detected and used to increase the confidence of detection of the target. In one embodiment, only overlapping fluorescence of DNA-specific labels and polynucleotide probes are identified as the detection of specific target sequences. In one embodiment, only site-specific labels that overlap an appropriate type-specific labels are accepted as true detection of the target site.

In one embodiment, excitation wavelength multiplexing is also used. Two or more excitation wavelengths are used to excite different set of labels. In one embodiment, type-specific labels and site-specific labels are chosen such that each can be excited by a different wavelength. Two images are then taken, one for each excitation. In one embodiment, type-specific labels and site-specific labels are excited using different wavelengths. In one embodiment, DAPI is used as the type-specific label and quantum dots are used as site-specific labels. In such an embodiment, 380 nm is used to excite DAPI and 570 nm can be used to excite the quantum dots.

In one embodiment, two or more recognition sites are located such that they can be resolved spatially. For example, two or more nucleotide sequences located on an elongated DNA molecule at distances between each other greater than the resolution of fluorescence microscopy. Other examples include surface antigens located on a bacterial cell at a distance greater than the spatial resolution of imaging method. In such an embodiment, spatial multiplexing can also be used. For example, the distance and/or order of different labels may be used as indication of detection of the pathogenic agent.

### 5.1.2. Detection of Labels

Detection of labels can be achieved using any method known in the art. In embodiments a sample is labeled with fluorescence labels, fluorescence microscopy can be used to achieve high spatial resolution detection.

The probes are preferably detected with a high spatial resolution. Preferably, the resolution is sufficiently high that labels in the same detection channel from different spatial locations on the surface are individually detectable. Subject to the technical limitation of the detection method used, the appropriate spatial resolution of detection can be selected according to the desired detection speed and the average spacing between labels which can be adjusted by labeling and washing. For example, when the surface density of detectable labels is small, the average spacing between labels is high, a detection method of lower spatial resolution may be used to increase detection speed. In one embodiment, when fluorescence labels are used, the surface is imaged by acquiring one or more images of the surface with a spatial resolution about the diffraction limit. In another embodiment, the spatial resolution is much finer than the size of the surface, e.g., at least 100, 1000, or 10,000 finer than the size of the surface.

The surfaces used to capture pathogenic agents are preferably small, e.g., between about 0.02 cm² and 2 cm², more preferably between about 0.1 cm² and 1 cm². Surface size can be chosen based on factors such as the volume of the sample to be evaluated and/or the resolution of the detection method, which depends on factors such as the density of residue non-specific binding and spatial resolution of the imaging device. In a preferred embodiment, optical microscopy is used as the detection method. The lateral resolution of far-field optical microscopy is determined by the diffraction limit, which is described by the Rayleigh length

$$δ x = \frac{0.61 λ}{N . A .} ,$$

where λ denotes the wavelength and N.A. denotes the numerical aperture of the objective: N.A.=5 n sin α, where n is the refractive index of the propagation medium and α is the half-aperture of the objective. Thus, the spatial resolution of far-field optical microscopy is approximately 150 nm for a wavelength of 500 nm and an N.A. of 1.4. The spatial resolution can be significantly increased by using near-field optical microscopy to about 20-100 nm. Thus, in one embodiment, the density of detectable labels on the surface including labeled pathogenic agents and non-specifically bound labels is less than about 0.2, 0.5, 1.0, 2.0, 10, 20, 50, 100, 500, or 1,000 per μm². In a preferred embodiment, far-field optical microscopy is used as the detection method, and the density of detectable labels on the surface including labeled pathogenic agents and non-specifically bound labels is less than about 0.2, 0.5, 1.0, 2.0, 10, 20, or 50 per μm2. In another preferred embodiment, near-field optical microscopy is used as the detection method, and the density of detectable labels on the surface including labeled pathogenic agents and non-specifically bound labels is less than about 50, 100, 500, or 1,000 per μm².

In one embodiment, fluorescence microscopy can be carried out using an inverted microscope, e.g., a Zeiss or Leica inverted microscopes. The magnification can be selected based on, e.g., the density of fluorescence molecules in the sample. Magnification can be 30×, 40×, 60×, 100×, and so on. In one embodiment, a 100× oil immersion objectives, numerical aperture 1.4 and a suitable band pass filter pack are used. Microscope images can be acquired using a suitable imaging device, e.g., a CCD imager. A video camera can also be attached to the microscope for visual inspection of the sample, and for examination of focus. A computer-controlled x-y microscope stage with a suitable translation resolution can be used for moving the sample. Excitation can be from any appropriate source, e.g., a laser of a suitable wavelength or a mercury arc lamp

In one embodiment, samples are imaged using a software routine which integrates all the microscope's functions such as the movement of the microscope stage, focus, and image collection. Digital images can be acquired by the microscope at a selected rate, e.g., 2 per min, and stored on hard disk arrays for later image processing and determination of degrees of colocalization.

In one embodiment, multiple emissions excited by a single excitation source, e.g., a single laser line, are separated using an appropriate means that splits the fluorescent light that has passed the confocal pinhole into its spectral components. Various optical methods can be used for this purpose. In one embodiment, an optical diffractive element, e.g., a grating, is used. These spectral components are projected onto a multi-channel detector, e.g., a detector consisting of a plurality of photo-multiplier elements, which collect photons across the whole detected spectrum. Parallel recording of the signals detected by these simultaneously illuminated elements results in a series of images of different wavelengths (“image stacks”) representing the spectral distribution of the fluorescence signals for every point of the confocal microscopic image.

These spectral images can be used for digital separation of the fluorescence emissions. This is based on linear comparisons of the spectral emission profiles with reference spectra characterizing the individual labels present in the sample. Reference spectra may either be derived from singly labeled control specimens and stored in a spectra database or directly taken from the experimental sample by selecting Regions of Interest. Start unmixing the signals by the click of a button. The result is a multi-channel image with every channel representing the quantitative distribution of an individual fluorochrome for every voxel in the image. Preferably, the diffraction element covers the whole range of wavelengths to be detected, e.g., the whole visible spectra and/or near infrared spectra, to allow sampling of emissions over the whole spectrum. Any fluorophore emissions in this range may be collected by electronic activation of the corresponding detector elements. Electronic selection not only guarantees stable recording, but also eliminates the need to sequentially step through individual bands to obtain an image stack. This reduces the total exposure to the exciting light and minimizes the detrimental effects of phototoxicity and photobleaching.

In another embodiment, signals are optically separated into channels defined by nonoverlapping spectral bands using a set of filters. An image is taken with each filter such that a set of images for different spectral bands are obtained.

In one embodiment, when excitation multiplexing is used, switching between excitation sources is used to achieve separation of signals from different excitation channels. Alternate scans are used to avoid the simultaneous excitation of and, hence, emission from the fluorophores. This is useful for those applications in which fluorophore combinations differ with respect to their excitation profiles.

### 5.1.3. Methods of Identifying a Pathogenic Agent Using Colocalization Analysis

In the method of the invention, detection of a pathogenic agent is based on the degree of colocalization of the set of labels used to probe the pathogenic agent. To identify a pathogenic agent, segments of data from the plurality of scanned images of the sample is analyzed. Colocalization of two or more labels is identified by colocalization analysis of the detection channels each corresponding to one of the labels (see, e.g., Manders, et al., 1993, Journal of Microscopy 169:375-382; Bio-Rad Technical Note 11; Media Cybernetics, Inc., Application Note #1).

In one embodiment, the degree of colocalization is measured from obtained multichannel images using an appropriate metric. Detection of the pathogenic agent is achieved by determining whether the metric is above a predetermined threshold value in the image or selected regions of the image.

In one embodiment, the total number or count of colocalization events detected is used as the metric. In one embodiment, the pathogenic agent is determined to be present in the sample if such total count is above 1, 2, 5, 10, 100, 1,000, or 10,000.

In another embodiment, Pearson's correlation coefficient of two fluorescence channels is used either alone or in combination with other colocalization metric to characterize the degree of colocalization of two different labels. Pearson's correlation coefficient can be calculated according to the following equation:

$${R}_{12} = \frac{{\sum }_{i} ( {S}_{1} ( i ) - {S}_{1}^{avg} ) \cdot ( {S}_{2} ( i ) - {S}_{2}^{avg} )}{{σ}_{1} \cdot {σ}_{2}} \qquad ( 3 )$$

where S₁(i) and S₂(i) are the signal intensities in the first and second channels, respectively, at the ith location, S₁^{avg} and S₂^{avg} are average signal intensities in the first and second channels, respectively, and σ₁ and σ₂ are standard deviations in the first and second channels, respectively. The normalization factor in the denominator in Eq. (3) ensures that Pearson's correlation coefficients are not dependent on the relative intensities of the fluorescent signals in the first and second channels or on the gain settings of the microscope's photodetectors. As can be deducted from Eq. (3), pixels that have a value that is strongly deviant from the average pixel value contribute most strongly to the value of R₁₂. In other words, the contribution of a given image location to the Pearson's correlation coefficient depends on its relative brightness within the image.

S₁^{avg} and S₂^{avg} can be an image wide average, region based averages, or a functional fit to the observed background levels. In one embodiment, σ₁ and σ₂ are calculated according to equation

$${σ}_{l} = \sqrt{{\sum }_{i} {( {S}_{l} ( i ) - {S}_{l}^{avg} )}^{2}} \qquad ( 4 )$$

where l=1 or 2. In another embodiment, σ₁ and σ₂ can be determined using an error model

$${σ}_{l} = \sqrt{{\sum }_{i} ( {{σ}_{l}^{bkg} ( i )}^{2} + {b}^{2} \cdot {S}_{l} ( i ) + {a}^{2} \cdot {{S}_{l} ( i )}^{2} )} \qquad ( 5 )$$

where σ₁^{bkg}(i) is an additive error of the ith pixel in the lth channel, a and b are coefficients. In one embodiment, b is set to zero, which gives a two-term error model. The additive error and coefficients in (5) can be determined according to U.S. Patent Publication No. 2003-0226098, which is incorporate herein by reference in its entirety.

Pearson's correlation coefficient has a value between −1 and 1, with −1 being no overlap between images and 1 being perfect image registration. Pearson's correlation coefficient takes into account only the similarity of objects' distribution and/or shapes between images and does not take into account image intensity. Since a negative value can be reported using this method, in one embodiment, other coefficients are used in combination with Pearson's correlation coefficient to characterize colocalization of different labels.

In another embodiment, an overlap coefficient is used either alone or in combination with other colocalization metric to characterize colocalization of different labels. The overlap coefficient has a value between 0 and 1. The overlap coefficient can be calculated according to the following equation:

$${R}_{12}^{oc} = \frac{{\sum }_{i} {S}_{1} ( i ) \cdot {S}_{2} ( i )}{\sqrt{{\sum }_{i} {( {S}_{1} ( i ) )}^{2} \cdot {\sum }_{i} {( {S}_{2} ( i ) )}^{2}}} \qquad ( 6 )$$

where S₁(i) and S₂(i) are defined as above, i.e., the signal intensities in the first and second channels, respectively, at the ith location.

In another embodiment, overlap coefficient k₁ and k₂ are used to characterize colocalization of different labels. These coefficients describe the differences in intensities of the two channels: the value k₁ is sensitive to differences in intensity for channel 1 while k₂ is sensitive to differences in intensity for channel 2. The overlap coefficient k₁ and k₂ can be calculated according to the following equations:

$$\begin{matrix}{k}_{1} = \frac{{\sum }_{i} {S}_{1} ( i ) \cdot {S}_{2} ( i )}{{\sum }_{i} {( {S}_{1} ( i ) )}^{2}} & ( 7 ) \\ {k}_{2} = \frac{{\sum }_{i} {S}_{1} ( i ) \cdot {S}_{2} ( i )}{{\sum }_{i} {( {S}_{2} ( i ) )}^{2}} & ( 8 )\end{matrix}$$

where S₁(i) and S₂(i) are defined as above, i.e., the signal intensities in the first and second channels, respectively, at the ith location.

In still another embodiment, colocalization coefficients m₁ and m₂ are used to characterize colocalization of different labels. These coefficients can be used to estimate the contribution of one color channel in the colocalized areas of the image to the overall colocalized fluorescence in the image: m₁ is used to describe the contribution of channel 1 to the colocalized area while m₂ is used to describe the contribution of channel 2. The overlap coefficient m₁ and m₂ can be calculated according to the following equations:

$$\begin{matrix}{m}_{1} = \frac{{\sum }_{i} {S}_{1}^{coloc} ( i )}{{\sum }_{i} {S}_{1} ( i )} & ( 9 ) \\ {m}_{2} = \frac{{\sum }_{i} {S}_{2}^{coloc} ( i )}{{\sum }_{i} {S}_{2} ( i )} & ( 10 )\end{matrix}$$

where S₁(i) and S₂(i) are defined as above, i.e., the signal intensities in the first and second channels, respectively, at the ith location, and
S₁^{coloc}(i)=S₁(i), if S₂(i)>0 (11)
S₂^{coloc}(i)=S₂(i), if S₁(i)>0 (12)
The coefficients generated are between zero and one. A value of zero means that there is no colocalization and a value of 1.0 means there is complete colocalization. As an example, a coefficient is generated for each color of the two colors in the pair of channels, e.g., Red 0.9 Green 0.45, would mean that the ratio of all the red intensities which showed a green component divided by the sum of all the red intensities in the selected area is 0.9, i.e. a very high degree of colocalization, and that the ratio of all the green intensities which showed a red component divided by the sum of all the green intensities is 0.45 which is half the colocalization value. So there is twice the degree of colocalization of red pixels with green as there is of green pixels with red.

In still another embodiment, colocalization coefficients M₁ and M₂ are used to characterize colocalization of different labels. M₁ is used to describe the contribution of channel 1 to the colocalized area while M₂ is used to describe the contribution of channel 2. The overlap coefficients M₁ and M₂ can be calculated according to the following equation:

$$\begin{matrix}{M}_{1} = \frac{{\sum }_{i} {S}_{1}^{coloc} ( i )}{{\sum }_{i} {S}_{1} ( i )} & ( 13 ) \\ {M}_{2} = \frac{{\sum }_{i} {S}_{2}^{coloc} ( i )}{{\sum }_{i} {S}_{2} ( i )} & ( 14 )\end{matrix}$$

where S₁^{coloc}(i)=S₁(i) if S₂(i) is within thresholds defined by area of interest or AOI (left and right sides of AOI in case of rectangular AOI), S₁^{coloc}(i)=0 if S₂(i) is outside the threshold levels. S₂^{coloc}(i)=S₂(i) if S₁(i) is within thresholds (top and bottom margins of AOI in case of rectangular AOI), S₂^{coloc}(i)=0 if S₁(i) is outside the AOI. These coefficients, M₁ and M₂, are proportional to the amount of fluorescence of colocalizing objects in each component of the image, relative to the total fluorescence in that component. The components are described as the channel 1 and channel 2 images, respectively.

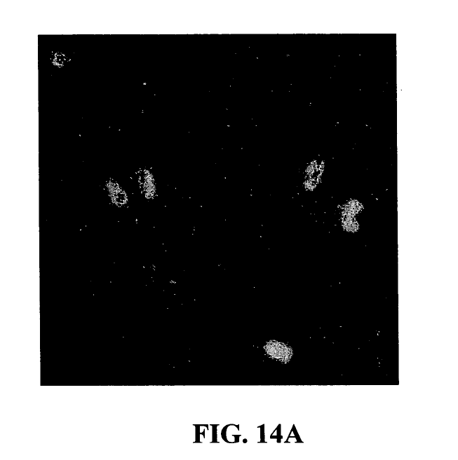

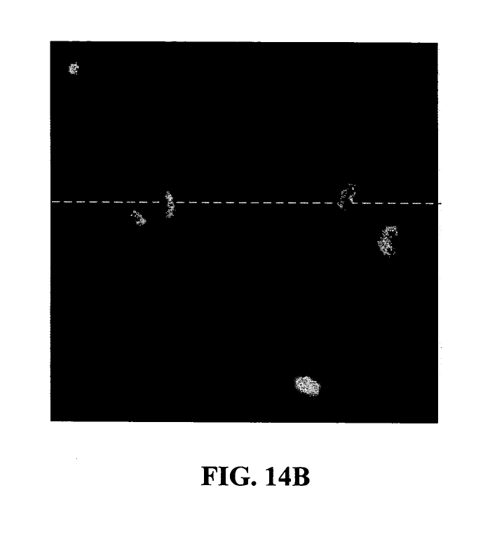

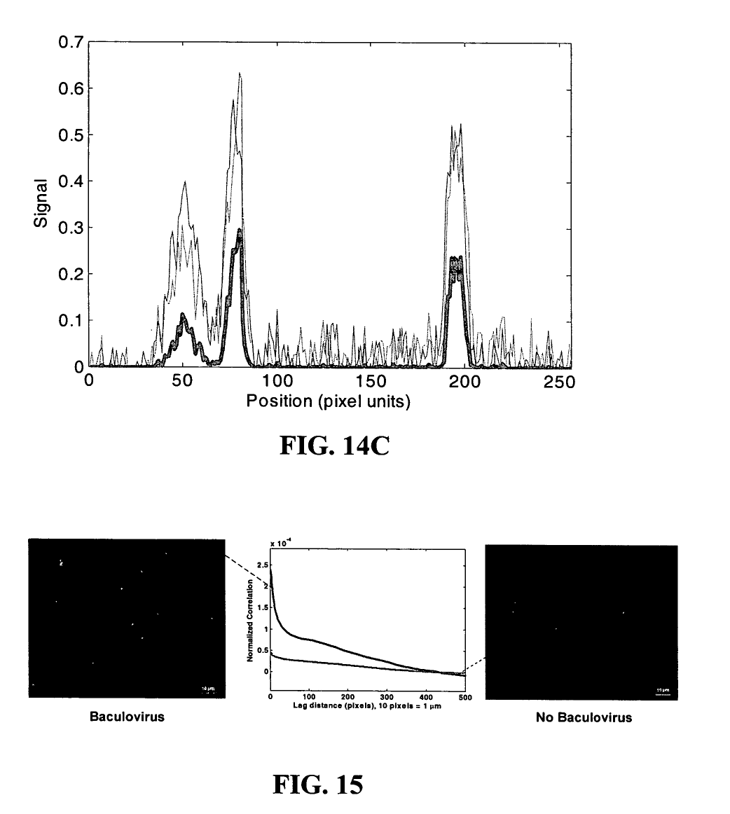

The feature detection step can operate on one or more cross-sections of the image, e.g., the cross-section corresponding to the bold trace in FIG. 14B, such as convolution with a template profile having the expected size and shape of a bacterium. The feature detection step can perform convolution in two dimensions. In another embodiment, the feature detection step can operate in Fourier space.

In another embodiment, the feature detection step performs thresholding in each channel, then look for the fraction of pixels where both channels are over threshold.

In other embodiments, where near-colocalization is to be detected, a statistical space-color covariance can be estimated. Pathogenic agents are detect by the peak in this covariance function near zero spatial lag. In one embodiment, an image detection region is selected and the average value of the product of the intensity in one channel at one location times the intensity in the other channel at another location is generated according to equation,
Cov=Avg over Region{I_{red}(x₁)I_{green}(x₂)} (15)
This results in a function of (x₁-x₂) that has a peak near zero lag if there are features where the labels colocalize.

Referring to FIG. 15, two different antibodies to Baculovirus gp64 surface protein were labeled with different quantum dot labels (here rendered as green and red fluorescent intensities). Incubation and wash were accomplished via the methods of the invention in 5 minutes and 1 minute, respectively. The probe concentration was 40 nM. The average product of intensities between the two colors at different positions (x, x+Δ) was computed via digital Fourier Transform correlation of the microscope image, and the resulting circularly symmetric correlation function was averaged over position angle to yield a function of distance only (graph), in accordance with the equations below. A control experiment with no target virus (right panel) yielded little increase at small lags (lower curve in graph), whereas with the target present (left panel) a sharp increase at small lags corresponding to the 1-5 micron particle sizes is apparent in the correlation function (upper curve in graph). Equations 16 and 17 below compute the spatial correlation between two image channels as a function of distance. When numerous detection sites, e.g., image features, are present some correlation will mostly likely be seen at all distance scales because at least one pair of features will be separated by a given distance. The strong correlation at short distances that are comparable to a cell diameter, is evidence for the two channel detection of individual features.
C(Δ)=∫d²×S_{A}(x)S_{B}(x+Δ)≈IFT[FT(S_{A}(x))FT*(S_{B}(x))] (16)
C(r)=(1/(2πr))∫dφC(r,φ) (17)

In one embodiment, methods using the distribution of interpoint distances (Ripley, 1980, Spatial statistics. John Wiley & Sons, New York, Chichester, Brisbane, Toronto; and http://nucleus.biomed.cas.cz/gold/IE/2.htm; http://nucleus.biomed.cas.cz/gold/IE/3.htm; http://nucleus.biomed.cas.cz/gold/IE/4.htm) is used for determining colocalization of labels. In the methods, functions characterizing the density of labels as a function of the distance from other labels is used to characterize the spatial statistics (Ripley, 1980, Spatial statistics. John Wiley & Sons, New York, Chichester, Brisbane, Toronto; and http://nucleus.biomed.cas.cz/gold/IE/2.htm; http://nucleus.biomed.cas.cz/gold/IE/3.htm; http://nucleus.biomed.cas.cz/gold/IE/4.htm). In one embodiment, to analyse the colocalization of different labels, the pair-correlation function (PCF) and the second reduced moment function (K function) is evaluated. In another embodiment, to analyse the colocalization of different labels, the pair cross-correlation function (PCCF) and the second reduced moment (or cross-K) function are used (Ripley, 1980, Spatial statistics. John Wiley & Sons, New York, Chichester, Brisbane, Toronto; and http://nucleus.biomed.cas.cz/gold/IE/2.htm; http://nucleus.biomed.cas.cz/gold/IE/3.htm; http://nucleus.biomed.cas.cz/gold/IE/4.htm).

In one embodiment, when a type-specific label is used, a type of pathogenic agents, e.g., cells or a type of cellular constituents, e.g., DNA, are identified from the obtained images by identifying objects labeled with the labels specific for the type of pathogenic agents. In one embodiment, fluorescence intensity in a channel corresponding to the label is used to identify a type of pathogenic agents. If the fluorescence intensity in a channel corresponding to the label is higher than a given threshold in an object in an image, the object is characterized as the pathogenic agents. In one embodiment, type-specific label is used to define region of interest (ROI) for determining degree of colocalization, i.e., degree of colocalization is only determined for such ROIs. Colocalization of site-specific and type-specific labels is preferably detected using a method for near-colocalization detection.

For three or more channel images, colocalization analysis can be carried out using the above described method(s) between two or more different pairs of channels to obtain coefficients for each such pair of color combinations. In one embodiment, an independent threshold is used for each pair of channels. Colocalization of all channels can be determined based on the set of independent thresholds. For example, colocalization can be assigned to locations in an image for which the Pearson's colocalization coefficient for each pair of channels is greater than a threshold specific for the pair of channels. Such a colocalization results may optionally be display in a colocalization map in which pixels corresponding to colocalization of a particular set of channels is identified by a particular color in the image. A 3D colocalization map can also be generated in which the z axis of the plot represents pixel frequencies to allow visual assessment of which combinations of color intensities are typified by the sample.

In one embodiment, the threshold value of the metric of the degree of colocalization is determined using one or more reference samples containing known numbers of copies a target pathogenic agent. Preferably, the threshold value is obtained using the same detection method. In one embodiment, a calibration curve of the threshold value as a function of the number of copies of the pathogenic agent is generated using a plurality of reference samples each containing a different number of copies of the pathogenic agent. A measurement of the metric in a sample can then be compared to the calibration curve to determine the presence and concentration of the target pathogenic agent in the sample. In another embodiment, statistical significance or the confidence level of the detection can also be determined.

In one embodiment, a plurality of different sets of colocalized recognition sites is detected. Recognition sites detected by different sets in the plurality are not be colocalized. In one embodiment, a plurality of sets of nucleic acid probes, each set containing two or more probes specifically binding to colocalized sequences but not colocalized with target sequences of other sets, are used. This can be achieved, for example, using sets of probes in which probes of each set bound to sequences located within a few kilobases in the target DNA, whereas probes in different sets bound to sequences located at least a few kilobases, e.g., more than 10 kb, more than 100 kb, etc. In another embodiment, a plurality of sets of probes, each set containing two or more probes specifically binding to a different type of cellular constituents are used. In one embodiment, one type is a nucleic acid, the other type is a protein.

Pathogen samples include blood, urine, sputum, stool, nasal swabs, and swabs from areas of localized infection, e.g., skin and soft tissue. The concentrations of different viruses and bacteria in a sample depend on particular pathogen as well as time histories and relative distributions in the various sample types. For example, Salmonella typhi levels in blood of typhoid patients varied from <1 to ˜300 cfu/ml with median levels in the 1 to 2 cfu/ml range (Wain et al., 1998, J Clin Microbiol 36, 1683-7). However, the total number of viable and non-viable organisms that could be detected with nucleic acid based tests would be higher by some ratio. In a study of AIDS patients with Mycobacterium bacteremia (Wong et al., B., 1985, Am J Med 78, 35-40), bacterial counts ranged from 350 to 28,000 cfu/ml. In wounds associated with bone fractures bacterial counts of 10⁵ per gram of tissue were observed (Sen et al., 2000, J Orthop Surg (Hong Kong) 8, 1-5). HIV levels in serum during the onset of AIDS can be 10⁴ to 10⁷ per ml (Schacker et al., 1998, Ann Intern Med 128, 613-20). Plasma levels of 10⁴-10⁶ per ml are seen in chronic HIV and HCV infections (Hawkins et al., 1997, J Clin Microbiol 35, 187-92; Hodinka, 1998, Clin Diagn Virol 10, 25-47). In a study of SARS virus detectability in retrospectively confirmed SARS patients (Drosten et al., 2004, J Clin Microbiol 42, 2043-7), quantitative RT-PCR tests determined that typical virus concentrations were ˜10⁶ copies/ml in sputum, ˜5×10⁴ copies/ml in stool, and ˜5×10² copies/ml in throat swabs and saliva. Samples from the lower respiratory tract gave the highest detection rate, where 12/12 samples were positive. Detectability vs. time since onset of SARS symptoms was studied by Chan, et al (Chan et al., 2004, Emerg Infect Dis 10, 825-31). Stool samples gave the highest detection rate, but this rate peaked two to three weeks after onset. Urine levels peaked after three or four weeks. In general, tissues that are the site of initial infection, and those that are most affected by a particular organism, will be the best targets for early detection.

A pathogenic agent is determined to be present in the sample if the set of one or more probes that bind the pathogenic agent is detected in the appropriate detection channels.

In one embodiment, a reference sample can be used for comparison with the sample to be tested. The reference sample can be a sample that does not comprise the pathogenic agent to be detected. This is useful to get the probe—surface binding. The reference sample can also comprise the pathogenic agent at predetermined amounts. The reference sample can be used to prepare the imaging surface in the same way as with the sample, and an image is taken. The sample image and the reference image can be compared, e.g., counts of a particular label in the sample and the reference images can be compared.

In one embodiment, a series of reference samples can be prepared, each having a different amount of one or more pathogenic agents of interest. Reference images are prepared and imaged to generate calibration curve. A sample can then be compared to the references.

In another embodiment, statistical significance or confidence level of detection of a pathogenic agent can be determined.

The sensitivity of the method is at least 100, more preferably 50, organisms per ml of sample for viruses and bacteria. The sensitivity of the method for toxins is at least 1000, more preferably 100, copies per ml. The number of labels used to detect each pathogen can be increased to increase the detection sensitivity and accuracy.

Using the method of the present invention, a sensitivity of at least 1,000, 500, 100, 50, 20, 10, 5, 2 or 1 organism per ml of sample can be achieved for viruses and bacteria, whereas a sensitivity of at least 10,000, 1,000, 500, 200, 100, 50, 20, 10 or 1 copy per ml of sample can be achieved for toxins.

In one embodiment, Probabilities of Detection (P_{D}) and Probabilities of False Positive (P_{FA}) on clinical samples are used to evaluate the performance of the methods. The synthetic samples contain known quantities of surrogate threat material, including the case of zero threat as negative control. For tests involving parallel detection of many agents, the sample contains only one or a few of the threats in non-zero quantity. False positives is assessed for the threats which were probed for but not included in the sample. A typical round of testing includes ˜20 independently created samples with ˜10 threats probed for in parallel. Thus false positive statistics is obtained for 20×10=200 threat hypotheses, which provides enough statistical stability to estimate P_{FA.} Tests are run at different spike-in levels to establish the lower limit of detection that can be achieved while maintaining a useful P_{D} and P_{FA}. The robustness to interfering human genomic DNA is also tested by adding known concentrations of human DNA. These tests establishes the following probabilities of detection and of false alarms at the lower limit of detection: P_{D}>0.95 averaged over the test organisms and P_{FA}<0.01 summed over all the threat hypotheses tested and averaged over the tests.

### 5.2. Methods of Detection

The methods of the invention can be used in conjunction with various types of detection methods. In one embodiment, the methods described in U.S. Provisional Patent Application No. to be assigned, Attorney Docket No. 11531-011-888, by Stoughton et al., filed on even date herewith, which is incorporated herein by reference in its entirety, are used. Blood, urine, sputum, stool, nasal swabs, and swabs from areas of localized infection, e.g., skin and soft tissue, all are likely sources for pathogen samples. For example, the diagnosis of anthrax (Bacillus anthracis) can involve visual microscopic recognition of the bacterial cells taken from skin lesions, serum, or nasal swab (Swartz, 2001, N Engl J Med 345, 1621-6). The concentrations of viruses and bacteria may have different time histories and relative distributions in the various sample types, and this behavior may be different for each pathogen. Salmonella typhi levels in blood of typhoid patients varied from <1 to ˜300 cfu/ml with median levels in the 1 to 2 cfu/ml range (Wain et al., 1998, J Clin Microbiol 36, 1683-7). However, the total number of viable and non-viable organisms that could be detected with nucleic acid based tests would be higher by some ratio. In a study of AIDS patients with Mycobacterium bacteremia (Wong et al., B., 1985, Am J Med 78, 35-40), bacterial counts ranged from 350 to 28,000 cfu/ml. In wounds associated with bone fractures bacterial counts of 10⁵ per gram of tissue were observed (Sen et al., 2000, J Orthop Surg (Hong Kong) 8, 1-5).

HIV levels in serum during the onset of AIDS can be 10⁴ to 10⁷ per ml (Schacker et al., 1998, Ann Intern Med 128, 613-20). Plasma levels of 10⁴-10⁶ per ml are seen in chronic HIV and HCV infections (Hawkins et al., 1997, J Clin Microbiol 35, 187-92; Hodinka, 1998, Clin Diagn Virol 10, 25-47). In a study of SARS virus detectability in retrospectively confirmed SARS patients (Drosten et al., 2004, J Clin Microbiol 42, 2043-7), quantitative RT-PCR tests determined that typical virus concentrations were ˜10⁶ copies/ml in sputum, ˜5×10⁴ copies/ml in stool, and ˜5×10² copies/ml in throat swabs and saliva. Samples from the lower respiratory tract gave the highest detection rate, where 12/12 samples were positive. Detectability vs. time since onset of SARS symptoms was studied by Chan, et al (Chan et al., 2004, Emerg Infect Dis 10, 825-31). Stool samples gave the highest detection rate, but this rate peaked two to three weeks after onset. Urine levels peaked after three or four weeks. In general, tissues that are the site of initial infection, and those that are most affected by a particular organism, will be the best targets for early detection.

Nasal swabs have been used in studies of Staphylococcus aureus (Paule et al., 2004, J Mol Diagn 6, 191-6), influenza (Bosis et al., 2005, J Med Virol 75, 101-4; Pregliasco et al., 2004, J Med Virol 73, 269-73), respiratory syncytial virus (Bosis et al., 2005, J Med Virol 75, 101-4), Metapneumovirus (Maggi et al., 2003, J Clin Microbiol 41, 2987-91), and several other respiratory diseases (Druce et al., 2005, J Med Virol 75, 122-9). Moderate to strong associations with disease were seen, suggesting that the nasal levels were primarily disease- and not exposure-related. However, nasal levels also can indicate exposure with or without infection. For example, nasal swabs were used to detect spores of the biologic insecticide Bacillus thuringiensis subsp. kurstaki HD1 pre- and post-agricultural aerial spraying in a Canadian safety study (Valadares De Amorim et al., 2001, Appl Environ Microbiol 67, 1035-43). This bacterial species is a close relative of B. anthracis, so detection of these spores after spraying provides a model for infectious disease investigations following a possible bioterrorism incident. In this study, the organism was detected in some nasal swabs before the study-associated spraying events, but the detection rate increased significantly after the aerosol release. The swabs were collected 2 hr after each of three different sprayings in the same general area. Nasal swabs are likely to be a key sample type in the near-term responses to suspected bioterrorism events. Sporulating bacteria may be present in ungerminated form in the nasal passage. These spores will require more rigorous lysis procedures to access the genomic material for DNA-based detection. There is however a significant amount of DNA associated with spore surfaces.

FIG. 1 shows an exemplary embodiment involving hybridization of the labeled probes to the target DNA occurs in solution. Alternatively, intact virions and bacteria can be captured on the filter, partially lysed and then labeled either with antibodies to surface proteins, or with DNA probes.

In some embodiments, separation and removal of human cells can be used to reduce the interference caused by the presence of large amounts of non-target human DNA and cell surface proteins. Lysis of bacterial cells and virions followed by hybridization with specific probes produces a mixture of bound and unbound probes. The unbound probes are separated via size exclusion to reduce the interfering signal from their fluorescent labels. Surprisingly, as will be shown below, efficient detection of individual labeled DNA fragments is readily possible using super-bright quantum dot fluorescent labels.

For blood samples, in one embodiment, host, e.g., human, cells are removed before detection of blood born pathogenic agents. This will reduce potential confusion of human and pathogen nucleic acid sequences caused by a much higher concentration of human sequences. Such host cell can, however, be used for additional detection. Some pathogens such as HIV, malaria, and human erythrovirus (Candotti et al., 2004, J Virol 78, 12169-78) exist within the human blood cells. Phagocytic cells also may contain pathogen DNA (Sanchez et al., 2004, J Virol 78, 10370-7). In a primate model of smallpox, disease was disseminated via monocytes (Jahrling et al., 2004, Proc Natl Acad Sci USA 101, 15196-200). In a mouse model of influenza (Mori et al., 1995, Microb Pathog 19, 237-44), viral RNA was detected in red blood cells from 1 to 5 days post-infection. Thus, in one embodiment, human cells are first removed from a serum sample, and are collected as a target for detection of pathogens existing in the human cells.

Effective lysis of bacterial spores, vegetative bacteria, and viruses can be achieved through a variety of methods. In one embodiment, an enzymatic or chemical method is used to lyse the organisms. In one embodiment, cells are lysed using a 0.5% SDS, 50 mM EDTA, 200 mM Tris, pH 7.4 solution,

In another embodiment, bead milling is used to disrupt sporulated and vegetative bacteria alone or in combination with an enzymatic or chemical method. Bead milling is advantageous in that only a few minutes of treatment are needed to effectively disrupt spores. In one embodiment, an acoustic based method is used for bacterial and viral lysis. The method utilizes agitation of a bead mixture through acoustic energy, yet not require integration of fast moving mechanical parts used in traditional bead milling (MicroFluidic Systems, Inc., MFSI, Pleasanton, Calif.). In addition to mechanical disruption provided by the beads, this system provides additional lysis efficiency from the acoustic energy.

In still another embodiment, an acoustic based lysis method without beads is used for cellular disruption through sonic induced cavitation events (Covaris, Inc., Woburn, Mass.). The method uses a transducer based megasonic technology which is effective at lysing cells for nucleic acid and protein extractions in tens of seconds. In another embodiment, the acoustic energy can also be scaled and tightly controlled to achieve fragmentation of the target DNA during cellular lysis.

In still another embodiment, lysis is carried out by capturing the intact microorganisms on a small pore size filter followed by treatment with a nonthermal plasma discharge to lyse the organisms directly on the filter (MicroEnergy Technologies, Inc., Vancouver, Wash.; and Atmospheric Glow Technologies, Inc., Knoxville, Tenn.). The plasma punches holes in the organisms, which holes are clearly seen in electron micrographs, making the nucleic acids and proteins in the cells available for labeling and detection. Cross-linking can be used to inhibit loss of the genomic material into solution. This method is particular useful in a labeling approach where the organisms are first immobilized on a filter and retain the spatial localization of their genomes during hybridization, essentially making the assay similar to fluorescence in situ hybridization (FISH). Quantum dot labeled probes have been used in the FISH modality to stain human metaphase chromosomes (Xiao et al., 2004, Nucleic Acids Res 32, e28). In one embodiment, the sample is treated for three minutes with plasma to permit recovery of intact DNA from bacterial spores.

A combination of two or more of the above methods can also be used to disrupt and lyse pathogens. In one embodiment, a combination of the following: physical methods such as bead milling or sonication; enzymatic methods such as proteinase K or lysozyme; and chemical methods employing detergents and/or chaotropic salts, is used to effectively lyse pathogens in a sample.

In the method of the invention, pathogenic agents and/or cellular constituents therefrom are captured on an appropriate surface. In one embodiment, the captured pathogenic agents and/or their cellular constituents are fixed on the surface. In one embodiment, the surface is the surface of a filter having an appropriate pore size. The pathogenic agents and/or cellular constituents therefrom are captured by passing the sample through the filter such that the pathogenic agents and/or cellular constituents therefrom are collected by the filter. In one embodiment, the filter captures and immobilizes the pathogenic agents. The pathogenic agents are then disrupted, i.e., lysed, to obtain the cellular constituents.

The surface containing the captured pathogenic agents and/or their cellular constituents is contacted with a probe composition that comprises a set of one or more probes that specifically bind a pathogenic agent of interest and/or cellular constituents therefrom under conditions that specific binding occurs. In a preferred embodiment, each of the probes in the probe composition has a concentration of at least 1 nM, 2 nM, 5 nM, 10 nM, 20 nM, 50 nM, or 100 nM. In another preferred embodiment, the concentration of each probe is selected such that specific binding of the probes to at least 10%, 20%, 30%, 50%, 70%, or 90% of their respective target recognition sites occurs within about 1, 2, 5, 10, or 15 minutes. Preferably, at least some of the probes in the probe composition are selected to have binding constants to their respective target recognition sites higher than a given specific binding threshold. In preferred embodiments, at least 10%, 20%, 50%, 70% 90%, or all probes in the probe composition have binding constants to their respective target recognition sites higher than a given specific binding threshold. Methods for selecting probes are described in Section 5.4., infra.

Each probe is labeled with a detectable label. In one embodiment, each probe is labeled with a fluorescence label, e.g., a fluorescence dye or a fluorescence quantum dot. Thus, the binding of the probes to recognition sites labels the recognition sites.

The method of the invention is preferably configured for detection of a plurality of different pathogenic agents in a sample in parallel. This is achieved by including a set of one or more probes for each of the pathogenic agents of interest in the probe composition. In one embodiment, the probe composition comprises a set of one or more probes for each of at least 5, 10, 20, 50, or 100 different pathogenic agents.

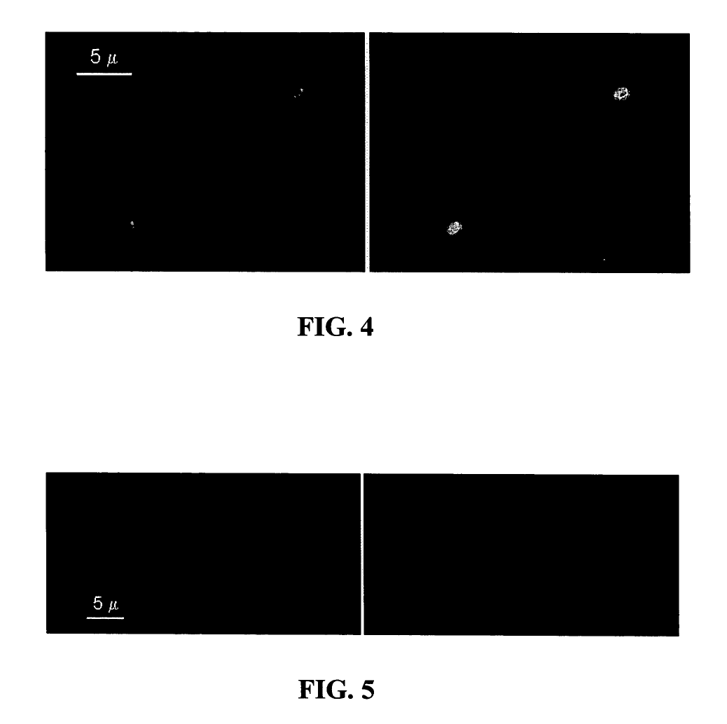

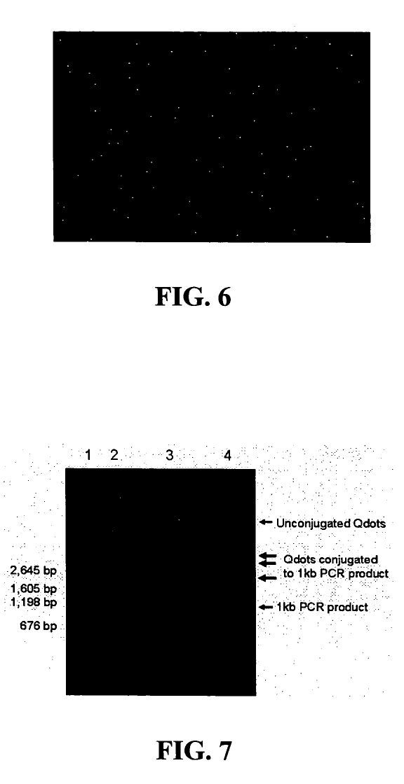

As illustrated in FIG. 4, by allocating to each pathogenic agent or threat organism two or more differently colored probes for different recognition sites, large gains in specificity and sensitivity can be obtained. As an illustration, allocating two different emission bands (colors) to each threat allows (6)(5)/2=15 threats or threat categories to be distinguished in a single reaction if fluorescence labels of 6 different emission bands are used for color coding. By grouping threats into categories according to the appropriate near-term response action, a diverse threat list could be covered in a single test. For example, hemorrhagic fever agents all could be given the same two-color code, since the immediate response action upon detection probably would be the same: namely, quarantine and confirmatory tests. Two approaches are available to provide finer resolution of threats. In the first approach, identification of the particular threat within a category will be accomplished with a second round of operation of the sensor using threat-unique labeling. Because of the system speed, this will add only a few minutes to the total timeline. The speed of the sensor will enable both rounds of confirmation to be completed in <20 min. In the second approach, combinations of labels colors can be combined into microspheres (Han et al., 2001, Nature Biotechnology 19:631-635) which then have a much greater potential dimensionality in color. This may allow an adequately large list of threats to be distinguished in one round of detection.

The use of high probe concentrations to achieve fast signal build up brings with it the problem of separating out the large number of unbound labeled probes prior to detection. The labeled surface can be washed with a wash composition to remove non-specifically bound probes. In a preferred embodiment, the wash composition dissociates probes that bind with a binding constant less than a given non-specific binding threshold. The non-specific binding threshold is preferably lower than the specific binding threshold. The non-specific binding threshold is preferably higher than binding of the probes to the surface. Thus, after the wash step, specific bound probes are retained, whereas probes non-specifically bound, e.g., bound to the surface, are removed. In a preferred embodiment, the non-specific binding threshold is fraction of the specific binding threshold. In one embodiment, the non-specific binding threshold is about 5%, 1%, 0.1%, 0.01% or 0.001% of the specific binding threshold. In another embodiment, the non-specific binding threshold is selected such that dissociation of at least a given percentage of the non-specifically bound probes occurs within a given wash time period. In one embodiment, the non-specific binding threshold is selected such that dissociation of at least half of the non-specifically bound probes occurs within about 15, 10, 5, or 1 minute, or about 30 or 10 seconds. In one embodiment, when a filter is used to capture pathogenic agents from the sample, the washing step can be carried out by contacting the filter surface with the wash composition for a given period of time and then remove the wash composition by passing it through the filter.

In one embodiment, ultrafiltration is used to pass unbound probes while retaining the probes that are bound to ˜kilobase or larger DNA fragments or large proteins. The large processing gains from high spatial resolution and color coincidence detection allow tolerance of a substantial residual number of unbound probes. In another embodiment, a flow-through geometry in which the target fragments are first immobilized on a surface is used. In still another embodiment, virions, bacterial cells, or spores are first immobilized on a filter. A partial lysis of the immobilized organisms is then carried out. The sample is then labeled with DNA labeling reaction. This allows using a courser filter that will permit more unbound label to escape. It also has the advantage that the identity of the organism is potentially more recognizable from the results of the labeling and imaging because the full complexity of the particular genome is retained at one spot.

Detection of labels can be achieved using any method known in the art. In embodiments a sample is labeled with fluorescence labels, fluorescence microscopy can be used to achieve high spatial resolution detection.

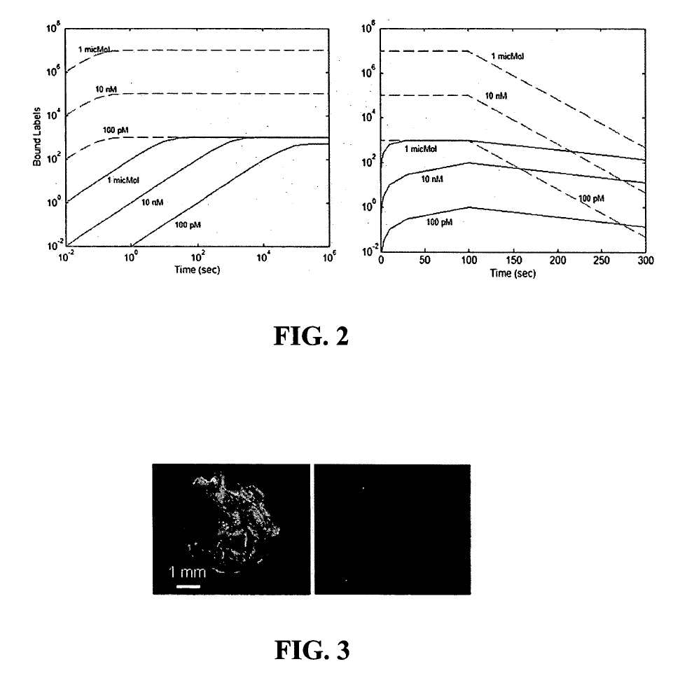

In one embodiment, nucleic acids are detected using polynucleotide probes. In order to accomplish fast detection without DNA amplification, labeling is accompolished a regime of binding kinetics different from that used in most molecular assays. Instead of allowing a low concentration of ligands to slowly find their correct binding sites, as in a -1 hour ELISA test or overnight microarray hybridization, a high ligand concentration is used to speed up the creation of duplexes. However, this results in a large amount of non-specific binding which must then be removed by a stringent denaturing. The resulting kinetics (Lauffenburger, D. A., and Linderman, J. J., 1993, Receptors: models for binding, trafficking, and signaling, Oxford University Press, New York) were simulated and are illustrated in FIG. 2 for a set of particular parameter choices. Some general features of the association and dissociation reactions are clear. For large ligand concentrations the approach to equilibrium during association is very fast, and above a certain ligand concentration signal saturates. During wash, although signal integrated over a large area is lost, there is a rapid increase in the ratio of signal to clutter.

Optimal hybridization and wash conditions for nucleic acid probes can be determined by a person skilled in the art. In general, it will depend on the length (e.g., oligomer versus polynucleotide greater than 200 bases) and type (e.g., RNA, or DNA) of probe and target nucleic acids. In one embodiment, the temperature and salt conditions (i.e., the “stringency”) of the hybridization or post-hybiridization washing conditions are selected to reduce non-specific binding. In one embodiment, “highly stringent” wash conditions are employed so as to destabilize all but the most stable duplexes such that hybridization signals are obtained only from the sequences that hybridize most specifically, and are therefore the most homologous, to the probe. Exemplary highly stringent conditions comprise, e.g., hybridization to filter-bound DNA in 5×SSC, 1% sodium dodecyl sulfate (SDS), 1 mM EDTA at 65° C., and washing in 0.1×SSC/0.1% SDS at 68° C. (Ausubel et al., eds., 1989, Current Protocols in Molecular Biology, Vol. I, Green Publishing Associates, Inc., and John Wiley & Sons, Inc., New York, N.Y., at p. 2.10.3). Alternatively, “moderate-” or “low-stringency” wash conditions may be used to allow detection of sequences which are related, not just identical, to the probe, such as members of a multi-gene family, or homologous genes in a different organism. Such conditions are well known in the art (see, e.g., Sambrook et al., supra; Ausubel, F. M. et al., supra). Exemplary moderately stringent wash conditions comprise, e.g., washing in 0.2×SSC/0.1% SDS at 42° C. (Ausubel et al., 1989, supra). Exemplary low-stringency washing conditions include, e.g., washing in 5×SSC or in 0.2×SSC/0.1% SDS at room temperature (Ausubel et al., 1989, supra).

The exact wash conditions that are optimal depend on the exact nucleic acid sequence or sequences of interest. Thus, in the present invention, probes having uniform specificity are preferably used. Such probes allows the use of one or a small number of wash conditions to remove non-specifically bound probes.

In DNA-based detection, it is not necessary to retain the intact genomic DNA for detection. As shown at the lower left of FIG. 1, DNA fragments can be detected. Color coincidence detection can be used on individual fragments. The probes can be selected to by complementary to sequences within a few kilobases. Individual DNA fragments can be detected readily when tagged with superbright labels such as quantum dots. This is shown in FIG. 5, where ˜kilobase DNA fragments were each tagged with one quantum dot using biotin-streptavidin binding. Exposures of less than one second are sufficient to provide signals well above the background image noise level, using the Leica DM6000B imaging system.

This single-fragment detection capability produces very high detection efficiency in the sense that most labeled fragments are seen. Detection is limited in theory only by the statistics of the number of target fragments present in the sample. It also enables color coincidence detection approach, in which two or more independent recognition sites separated by less than the DNA fragment size (a few kilobases or less) will be assigned probes with different colors. Detection of a specific target type will be declared only when both colors are present in an image pixel (see, e.g., Section 5.3.). Colocalization detection of two or more differently labeled DNA hybridization probes was done in a flow cell configuration (Castro et al., 1997, Anal Chem 69, 3915-20) in 1997 and was shown to provide dramatic processing gains that enabled specific detection of individual target fragments.

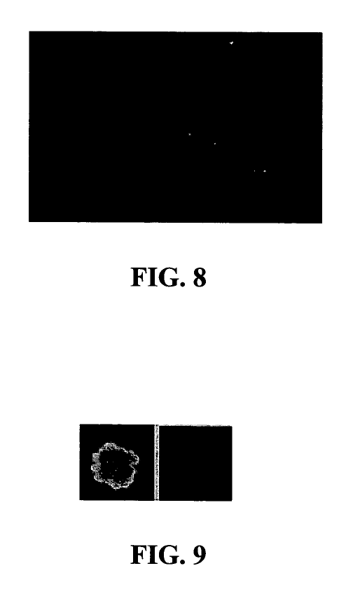

Gel electrophoresis was used to obtain and verify isolation of dot-labeled DNA from free dots (FIG. 7). This assay also is being used to monitor hybridization products in solution between Qdot-labeled probes and target DNA so that they can be related to their appearance under fluorescence microscopy. FIG. 8 shows a mix of unbound Qdot-labeled probes, 1-kb PCR products containing complementary binding sequences for the probes, and probes specifically duplexed to the 1-kb pieces. SYBR green staining of the double stranded DNA is rendered blue and shows up along a curvilinear structure which seems to be a chain of duplexes and 1-kb fragments made possible by the fact that multiple oligos are conjugated to each Qdot via its multiple streptavidin sites. A two minute hybridization time was used.

In another embodiment, one or more protein markers, e.g., surface antigens, are detected using antibodies that bind the markers.

As an illustration, FIG. 3 shows gp64 antibody to baculovirus surface protein was used to rapidly and specifically label baculovirus virions that had been captured on a 0.2 μ pore filter. In this experiment the non-specific binding of gp64 to the filter, and of the mismatched negative control antibody to the virions in the control experiment, was washed away through the filter with a stringent 10 sec wash. In this experiment 10⁵-10⁶ virions were present on the filter. For a more dilute sample, as was assumed in generating FIG. 2, total clutter signal may still exceed total specific signal after wash, as indicated in the right part of the right frame of FIG. 2. This can be circumvented by using high resolution imaging and color coincidence detection to greatly increase the effective signal to clutter ratio.

The gain derived from resolution is a familiar concept, illustrated in FIG. 4 where two E. coli cells were stained with quantum-dot labeled antibodies in a two minute incubation. Antibodies labeled with 605 nm emission dots and antibodies labeled with 705 nm emission dots were used together. The (unfiltered) solution was imaged under cover slip with our Leica DM6000B fluorescence imaging system. The individual unbound dot-labeled antibodies are clearly seen as a granular background in both color channels. Individual quantum dots also are seen bound to the cells via the antibodies. In both color channels there is a significant total brightness in the distributed background due to the unbound probes. However, the spatial resolution makes the detection of the cells obvious, and the fact that red and green labels only tend to collocate on the cells makes the detection even stronger; basing detection on yellow (coincident) pixels only, there would be essentially zero background. The actual gain from color coincident detection involves the degree of spatial correlation (lumpiness) of the background and how these lumps correlate between the color channels. This principle holds even when the target itself is smaller than a resolution cell (pixel) of the imaging system, as will be true for most viruses and individual DNA fragments. Thinking of non-target organisms as background, color coincidence enhances detection performance because the non-target organisms, even though they may be related biologically to the target organism, are much less likely to bind both of two different probes that were designed to be specific for the target organism.

In these antibody binding experiments, adequate signal for detection built up in less than one minute, and was E. coli specific (FIG. 5). As expected from FIG. 2, detectable signal accumulated faster when higher probe concentrations were used; detections were possible within ˜5 sec when using micromolar antibody titers.

### 5.3. Selection and Preparation of Probes

The probes for specific binding of particular recognition sites can be selected using methods known in the art. For a given target pathogenic agent, nucleic acid probes can be selected based on the genomic sequence of the pathogenic agent as described in Section 5.4.2. Probes that bind epitopes of proteins or toxins can be selected by various methods including methods described in Section 5.4.3.

### 5.3.1 Infectious Microorganisms

The methods of the invention can be used to detect infectious microorganisms of any kinds. Nucleic acid probes and/or antibody probes specific to an infectious microorganism are selected and used to determine whether such microorganism is present in a sample.

Viruses that can be detected include but are not limited to: influenza virus, human respiratory syncytial virus, Dengue virus, measles virus, herpes simplex virus type 2, poliovirus I, HIV I, hepatitis B, pseudorabies virus, transmissible gastroenteritis, swine rotavirus, swine parvovirus, bovine diarrhea virus, Newcastle disease virus, foot and mouth disease virus, hog colera virus, swine influenza virus, African swine fever virus, infectious bovine rhinotracheitis virus, infectious laryngotracheitis virus, La Crosse virus, neonatal calf diarrhea virus, Venezuelan equine encephalomyelitis virus, punta toro virus, murine leukemia virus, mouse mammary tumor virus, equine influenza virus or equine herpesvirus, bovine respiratory syncytial virus or bovine parainfluenza virus, bovine diarrhea virus, hepatitis virus type A, hepatitis type C, influenza, varicella, adenovirus, herpes simplex type I (HSV-I), herpes simplex type II (HSV-II), rinderpest, rhinovirus, echovirus, rotavirus, respiratory syncytial virus, papilloma virus, papova virus, cytomegalovirus, echinovirus, arbovirus, hantavirus, coxsachie virus, mumps virus, measles virus, rubella virus, polio virus, human immunodeficiency virus type I (HIV-I), and human immunodeficiency virus type II (HIV-II), any picornaviridae, enteroviruses, caliciviridae, any of the Norwalk group of viruses, togaviruses, such as Dengue virus, alphaviruses, flaviviruses, coronaviruses, rabies virus, Marburg viruses, ebola viruses, parainfluenza virus, orthomyxoviruses, bunyaviruses, arenaviruses, reoviruses, rotaviruses, orbiviruses, human T cell leukemia virus type I, human T cell leukemia virus type II, simian immunodeficiency virus, lentiviruses, polyomaviruses, parvoviruses, Epstein-Barr virus, human herpesvirus-6, cercopithecine herpes virus I (B virus), and poxviruses

Bacteria include, but are not limited to, Mycobacteria rickettsia, Mycoplasma, Neisseria spp. (e.g., Neisseria menigitidis and Neisseria gonorrhoeae), Legionella, Vibrio cholerae, Streptococci, such as Streptococcus pneumoniae, Corynebacteria diphtheriae, Clostridium tetani, Bordetella pertussis, Haemophilus spp. (e.g., influenzae), Chlamydia spp., enterotoxigenic Escherichia coli, and Bacillus anthracis (anthrax), etc.

Protozoa include, but are not limited to, plasmodia, eimeria, Leishmania, and trypanosoma.

In another embodiment, the method is used for detecting a toxin or drug in a sample. The toxin or drug can be a chemical or biological, e.g., venom. Envenomation by reptiles or insects often leads to the deposition of a mixture of toxic substances into the blood stream of the victim. The toxic substances in such a mixture are structurally heterogenous. The clinical symptom, i.e., poisoning, is a result of multiple blood-borne toxins.

In one embodiment the invention provides a method for detecting National Institute of Allergy and Infectious Diseases (NIAID) Category A, B and/or C priority pathogens. Category A includes Bacillus anthracis (anthrax); Clostridium botulinum; Yersinia pestis; Variola major (smallpox) and other pox viruses; Francisella tularensis (tularemia); Viral hemorrhagic fevers; Arenaviruses; LCM, Junin virus, Machupo virus, Guanarito virus; Lassa Fever; Bunyaviruses; Hantaviruses; Rift Valley Fever; Flaviruses; Dengue; Filoviruses; Ebola; and Marburg.

Category B includes Burkholderia pseudomallei; Coxiella burnetii (Q fever); Brucella species (brucellosis); Burkholderia mallei (glanders); Ricin toxin (from Ricinus communis); Epsilon toxin of Clostridium perfringens; Staphylococcus enterotoxin B; Typhus fever (Rickettsia prowazekii); Food and Waterborne Pathogens, including bacteria (Diarrheagenic E.coli, Pathogenic Vibrios, Shigella species, Salmonella, Listeria monocytogenes, Campylobacter jejuni, and Yersinia enterocolitica), viruses (Caliciviruses, Hepatitis A); and Protozoa (Cryptosporidium parvum, Cyclospora cayatanensis, Giardia lamblia, Entamoeba histolytica, Toxoplasma, Microsporidia); and additional viral encephalitides (West Nile Virus, LaCrosse, California encephalitis, VEE, EEE, WEE, Japanese Encephalitis Virus, Kyasanur Forest Virus).

Category C includes emerging infectious disease threats such as Nipah virus and additional hantaviruses, and NIAID priority areas: Tickborne hemorrhagic fever viruses, Crimean-Congo Hemorrhagic fever virus, Tickborne encephalitis viruses, Yellow fever, Multi-drug resistant TB, Influenza, Other Rickettsias, Rabies, and Severe acute respiratory syndrome-associated coronavirus (SARSCoV).

### 5.3.2. Pathogen Genomics and Selection of DNA Probes

In designing probes there is a fundamental conflict between the goal of differentiating closely related species and the need to detect strain variants whose sequences are not known at the time of probe design. Sequence regions and motifs that tend to be conserved across a clade tend to make robust targets but do not discriminate between organisms within the clade. In one embodiment, probes to sequences which are conserved within the group of organisms sharing the same phylogeny and pathogenic potential, but are not present in other organisms are selected. The implementation of this approach differs depending on the degree of sequence conservation expected. In addition, certain virulence gene cassettes, drug resistance markers, and even signature sequences related to deliberate bioengineering can be identified independently from a target pathogen. For example, a virulent strain of B. cereus recently was found to possess a plasmid very similar to the pX01 plasmid of B. anthracis (Hoffmaster et al., 2004, Proc Natl Acad Sci USA 101, 8449-54; Miller et al., 1997, J Clin Microbiol 35, 504-7).

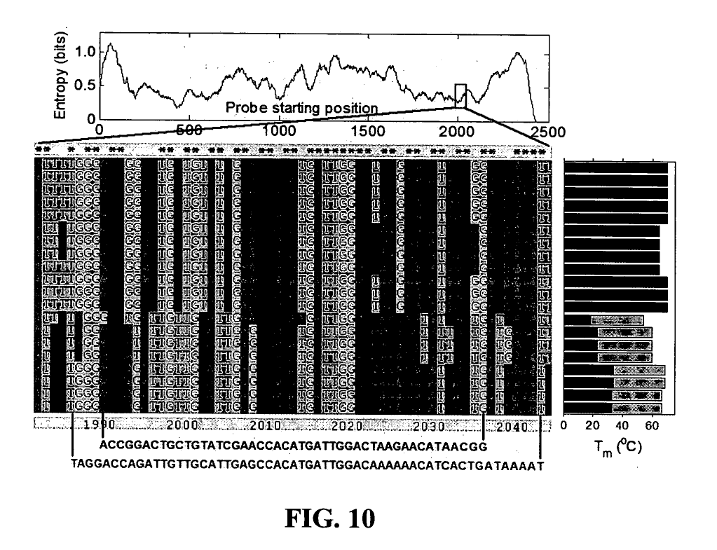

Many of the RNA viruses are highly mutative. Conservation between subtypes is poor at the nucleotide level. However, conserved regions for probe binding generally can be identified for the most conserved genes. FIG. 10 shows a conserved region of the envelope glycoprotein gene of Ebola Zaire, and the consensus probe sequences derived for it. Although many of the nucleotide positions are not conserved (gaps in asterisks at top of alignment), it is possible to find a workable probe sequence for each of two strain subgroups. FIG. 10 is an example of the output of the existing bioinformatics analysis pipeline.

The effective level of conservation as seen by the DNA probe can be increased by using chemically modified nucleotides, such as the ‘wild card’ deoxyinosine (Martin et al., 1985, Nucleic Acids Res 13, 8927-38; Napier et al., 1997, Bioconjug Chem 8, 906-13), which contributes less mismatch penalty than does a natural A, G, C, or T.

DNA viruses generally have a degree of conservation closer to that of bacteria than to that of the RNA viruses (Drake, 1999, Ann N Y Acad Sci 870, 100-7), making it fairly easy to design probes that will bind to all strains within a pathogenic group.

Bacterial genomes, although relatively stable compared to RNA viral genomes, include point mutations, insertions, deletions, cassettes, transposons, insertion elements and plasmids related to virulence that can vary within a species and even be traded between species. Virulence-associated sequences make good targets since their presence is directly related to clinical consequences in human infection. In one embodiment, the assay for B. anthracis

based on three genetic markers: one each for the pX01 and pX02 plasmids, and one for the spore structural gene sspE can be used. In one embodiment, exhaustive cross-reactivity studies on 11 B. anthracis strains and 29 related near-neighbor organisms within the same lade, which includes B. cereus and B. thuringiensis, can be performed to provide a robust and specific assay for virulent B. anthracis strains.

Successfully differentiated organisms within the anthracis clade can be achieved by identifying unique sequences scattered throughout their genomes without recourse to the plasmids. This approach was validated by hybridization to DNA microarrays containing these unique probes. When the relations of particular genes to virulence have not been established for a target organism, but a large fraction of its genomic sequence is available, this approach is particularly attractive.

The bioinformatics efforts will include developing the architecture of the database, development of algorithms for finding optimal DNA probe sequences, and development of software associated with actual operation of the device. The database development effort will continue throughout the program as a greater diversity of threats is addressed and as more sequence information becomes available.

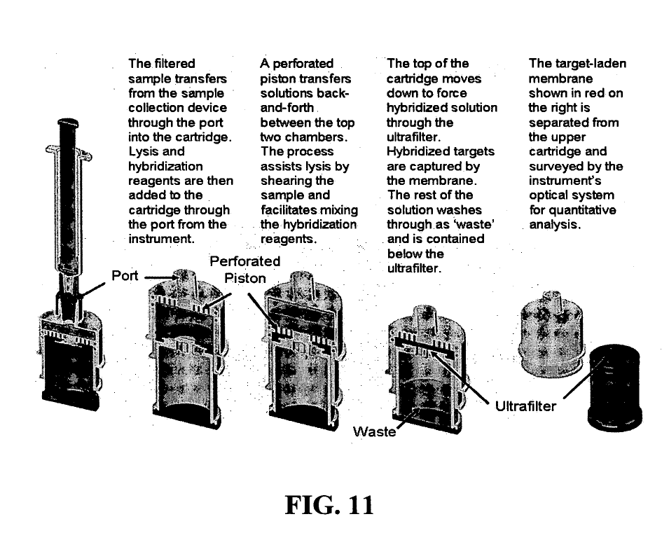

In one embodiment, probe sets are designed based on pathogens of interests and operational scenarios that the test is used. Exemplary choices for these probe sets are indicated in FIG. 11 and include a set for parallel detection of all Category A agents, a set for detection and detailed discrimination of B. anthracis strains and other near-neighbor organisms in that clade, and a set for detection and detailed discrimination of RNA viruses. Additional probe sets can be added. These probe reagent sets are also provided in kits for delivery.

Genome sequence information can be retrieved from several sources including NCBI, individual databases being developed under the NIAID Bioinformatics Resource Centers for Biodefense and Emerging or Re-Emerging Infectious Diseases program (NIAID. NIAID Bioinformatics Resource Centers for Biodefense and Emerging or Re-Emerging Infectious Diseases Program, http://www.niaid.nih.gov/dmid/genomes/brc/default.htm). An informnatics infrastructure is assembled including a database of genomic sequence representing Category A, B, and C pathogens and strain variants, probe design algorithms, and software linking the two.

Where possible, target recognition sequences for each threat organism will be chosen that are intimately related to its specific known virulence properties and mechanisms, as in the approach to the B. anthracis clade (Kim et al., 2005, FEMS Immunology and Medical Microbiology 43:301-310). In another embodiment, a detailed phylogenetic analysis of the clade surrounding each threat organism will be done to identify likely near-neighbor false positives and a biological basis for the choice of gene regions most likely to provide robust and specific detection (see, e.g., Kim et al., 2005, FEMS Immunology and Medical Microbiology 43:301-310).

In one embodiment, probe design in our approach involves choosing two or more identification sites per target sequence for oligo probe binding where these sites are separated by ˜5000 nucleotides or less to support spatial coincidence detection. At the same time, each label type can be assigned to several probes targeting different recognition sites widely separated over the genome, creating even more robust detection. In one embodiment, commercial softwares (e.g., ArrayDesigner, by Premier Biosoft International; TILIA, by Linden Biosciences) and public software (Li et al., 2001, Bioinformatics 17, 1067-76) are used for designing hybridization probes.

The probes need not be of the same length. In preferred embodiments, probes having uniform binding constant, e.g., constant Tm, but not having the same length are selected. In one embodiment, probe length is varied around 30 nucleotides to achieve roughly constant Tₘ so that an optimal trade between sensitivity and specificity can be made simultaneously for multiple probes. Tₘ can be computed based on a nearest neighbor model of solution phase oligo hybridization with quartet energy coefficients taken from published values for perfect match and mismatch quartets (SantaLucia et al., 1996, Biochemistry 35, 3555-62; SantaLucia et al., 1997, Biopolymers 44, 309-19; Sugimoto et al., 1995, Biochemistry 34, 11211-6; Sugimoto et al., 1996, Nucleic Acids Res 24, 4501-5). The steric effects of quantum dots on the hybridization can also be evaluated for refinement of the probe design rules. Probes can further be selected to avoid sequences with propensity for secondary structure, avoid low-complexity sequence, and avoid cross-hybridization to other targets. The cross-hybridization calculation is a computationally demanding but important part of the process. It can also consider the possible presence of other common infectious agents not on the NIAID Category A, B, C lists such as adenoviruses, rotoviruses, and common influenzas associated with upper respiratory and flu-like symptoms. It also will consider commensal organisms that often are carried without overt disease. Examples of these agents include (Heritage, 2003, The Human Commensal Flora, Leeds University Website) Herpesvirus simplex 1 (HSV1) associated with cold sores in the mouth mucosa, Streptococcus mutans associated with placque and tooth decay, Staphylococcus aureus often carried in the nose, Streptococcus pneumoniae, Streptococcus pyogenes and Neisseria meningitides often found in the throat. For nasal swabs, environmental background organisms need to be considered. These are potentially more diverse than those actually growing in the nasal passage, and include pollens and common airborne environmental bacteria such as Bacillus subtilis, Bacillus cereus, Bacillus thuringiensis, Burkholderia cepacia, and Ralstonia solanacearum. In particular, B. cereus and B. thuringiensis both are very close relatives of B. anthracis and are distinguished carefully in the probe design as described above. In one embodiment, polynucleotide probes having specificity and sensitivity above given threshold levels can be selected using the methods disclosed in WO01/05935, which is incorporated by reference herein in its entirety.

In operation scenarios where symptoms provide prior information, a probe composition can include probes for a panel of infectious agents that may cause the symptom. The sequence database will be augmented with the genomes of these common and commensal agents.

The methods of the present invention can be performed using any suitable nucleic acid probe or probes. For example, the probes may comprise DNA sequences, RNA sequences, or copolymer sequences of DNA and RNA. The probes may also comprise DNA and/or RNA analogues, or combinations thereof. For example, the polynucleotide probes may be full or partial sequences of genomic DNA, cDNA, or mRNA sequences extracted from cells. The polynucleotide probes may also be synthesized nucleotide probe, such as synthetic oligonucleotide probes. The probe sequences can be synthesized either enzymatically in vivo, enzymatically in vitro (e.g., by PCR), or non-enzymatically in vitro.

In one embodiment, the probes comprise nucleotide sequences greater than about 250 bases in length corresponding to one or more sequences in the genome or a transcript thereof in the target organism. For example, the probes may comprise DNA or DNA “mimics” (e.g., derivatives and analogues) corresponding to at least a portion of each gene in an organism's genome. In another embodiment, the probes are complementary RNA or RNA mimics. DNA mimics are polymers composed of subunits capable of specific, Watson-Crick-like hybridization with DNA, or of specific hybridization with RNA. The nucleic acids can be modified at the base moiety, at the sugar moiety, or at the phosphate backbone. Exemplary DNA mimics include, e.g., phosphorothioates. DNA can be obtained, e.g., by polymerase chain reaction (PCR) amplification of gene segments from genomic DNA, cDNA (e.g., by RT-PCR), or cloned sequences. PCR primers are preferably chosen based on known sequence of the genes or cDNA that result in amplification of unique fragments (i.e., fragments that do not share more than 10 bases of contiguous identical sequence with any other sequences in the genome of the organism). Computer programs that are well known in the art are useful in the design of primers with the required specificity and optimal amplification properties, such as Oligo version 5.0 (National Biosciences). Typically each such probe on the microarray will be between 20 bases and 50,000 bases, and usually between 300 bases and 1,000 bases in length. PCR methods are well known in the art, and are described, for example, in Innis et al., eds., 1990, PCR Protocols: A Guide to Methods and Applications, Academic Press Inc., San Diego, Calif. It will be apparent to one skilled in the art that controlled robotic systems are useful for isolating and amplifying nucleic acids. In other embodiments, the probes are made from plasmid or phage clones of genes, cDNAs (e.g., expressed sequence tags), or inserts therefrom (Nguyen et al., 1995, Genomics 29:207-209).

Polynucleotide probes can also be generated by synthesis of synthetic polynucleotides or oligonucleotides, e.g., using N-phosphonate or phosphoramidite chemistries (Froehler et al., 1986, Nucleic Acid Res. 14:5399-5407; McBride et al., 1983, Tetrahedron Lett. 24:246-248). Synthetic sequences are typically between about 15 and about 500 bases in length, more typically between about 20 and about 100 bases, most preferably between about 40 and about 70 bases in length. In some embodiments, synthetic nucleic acids include non-natural bases, such as, but by no means limited to, inosine. As noted above, nucleic acid analogues may be used as probes. An example of a suitable nucleic acid analogue is peptide nucleic acid (see, e.g., Egholm et al., 1993, Nature 363:566-568; U.S. Pat. No. 5,539,083).

### 5.3.3. Selection of Antibody Probes

Antibodies can be prepared by immunizing a suitable subject with an antigen or a fragment thereof as an immunogen. The antibody titer in the immunized subject can be monitored over time by standard techniques, such as with an enzyme linked immunosorbent assay (ELISA) using immobilized polypeptide. If desired, the antibody molecules can be isolated from the mammal (e.g., from the blood) and further purified by well-known techniques, such as protein A chromatography to obtain the IgG fraction.

At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975, Nature 256:495-497), the human B cell hybridoma technique by Kozbor et al. (1983, Immunol. Today 4:72), the EBV-hybridoma technique by Cole et al. (1985, Monoclonal Antibodies and Cancer Therapy, Alan R. Liss, Inc., pp. 77-96) or trioma techniques. The technology for producing hybridomas is well known (see Current Protocols in Immunology, 1994, John Wiley & Sons, Inc., New York, N.Y.). Hybridoma cells producing a monoclonal antibody of the invention are detected by screening the hybridoma culture supernatants for antibodies that bind the polypeptide of interest, e.g., using a standard ELISA assay.

Monoclonal antibodies are obtained from a population of substantially homogeneous antibodies, i.e., the individual antibodies comprising the population are identical except for possible naturally occurring mutations that may be present in minor amounts. Thus, the modifier “monoclonal” indicates the character of the antibody as not being a mixture of discrete antibodies. For example, the monoclonal antibodies may be made using the hybridoma method first described by Kohler et al., 1975, Nature, 256:495, or may be made by recombinant DNA methods (U.S. Pat. No. 4,816,567). The term “monoclonal antibody” as used herein also indicates that the antibody is an immunoglobulin.

In the hybridoma method of generating monoclonal antibodies, a mouse or other appropriate host animal, such as a hamster, is immunized as hereinabove described to elicit lymphocytes that produce or are capable of producing antibodies that will specifically bind to the protein used for immunization (see, e.g., U.S. Pat. No. 5,914,112, which is incorporated herein by reference in its entirety).

Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)). The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 cells available from the American Type Culture Collection, Rockville, Md. USA.

Human myeloma and mouse-human heteromyeloma cell lines also have been described for the production of human monoclonal antibodies (Kozbor, 1984, J. Immunol., 133:3001; Brodeur et al., Monoclonal Antibody Production Techniques and Applications, pp. 51-63 (Marcel Dekker, Inc., New York, 1987)). Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immuno-absorbent assay (ELISA). The binding affinity of the monoclonal antibody can, for example, be determined by the Scatchard analysis of Munson et al., 1980, Anal. Biochem., 107:220.

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103, Academic Press, 1986). Suitable culture media for this purpose include, for example, D-MEM or RPMI-1640 medium. In addition, the hybridoma cells may be grown in vivo as ascites tumors in an animal. The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Alternative to preparing monoclonal antibody-secreting hybridomas, a monoclonal antibody directed against an antigen or a fragment thereof can be identified and isolated by screening a recombinant combinatorial immunoglobulin library (e.g., an antibody phage display library) with the antigen or the fragment. Kits for generating and screening phage display libraries are commercially available (e.g., Pharmacia Recombinant Phage Antibody System, Catalog No. 27-9400-01; and the Stratagene antigen SurfZAP™ Phage Display Kit, Catalog No. 240612). Additionally, examples of methods and reagents particularly amenable for use in generating and screening antibody display library can be found in, for example, U.S. Pat. Nos. 5,223,409 and 5,514,548; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734.

The probe can also be an antigen-binding antibody fragment. An antigen-binding fragment can be produced by various methods known in the art.

In one embodiment, the antibody fragment is a fragment of an immunoglobulin molecule containing a binding domain which specifically binds an antigenic molecule. Examples of immunologically active fragments of immunoglobulin molecules include but are not limited to Fab, Fab′ and (Fab′)₂ fragments which can be generated by treating an appropriate antibody with an enzyme such as pepsin or papain. In a preferred embodiment, an antigen-binding antibody fragment is produced from a monoclonal antibody having the desired antigen binding specificity. Such a monoclonal antibody can be raised using the targeted antigenic molecule by any of the standard methods known in the art. For example, a monoclonal antibody directed against an antigenic molecule can be raised using any one of the methods described in Section 5.2.1., supra, using the antigenic molecule in the place of CR1. The antibody can then be treated with pepsin or papain. For example, pepsin digests an antibody below the disulfide linkages in the hinge region to produce an (Fab′)₂ fragment of the antibody which is a dimer of the Fab composed of a light chain joined to a V_{H}—C_{H}1 by a disulfide bond. The (Fab′)₂ fragments may be reduced under mild conditions to break the disulfide linkage in the hinge region thereby converting the (Fab′)₂ dimer to a Fab′ monomer. The Fab′ monomer is essentially an Fab with part of the hinge region. See Paul, ed., 1993, Fundamental Immunology, Third Edition (New York: Raven Press), for a detailed description of epitopes, antibodies and antibody fragments. A skilled person in the art will recognize that such Fab′ fragments may be synthesized de novo either chemically or using recombinant DNA technology. Thus, as used herein, the term antibody fragments includes antibody fragments produced by the modification of whole antibodies or those synthesized de novo.

In another embodiment, the method of generating and expressing immunologically active fragments of antibodies described in U.S. Pat. No. 5,648,237, which is incorporated herein by reference in its entirety, is used.

In still another embodiment, the antigen-binding antibody fragment, e.g., an Fv, Fab, Fab′, or (Fab′)₂ is produced by a method comprising affinity screening of a phage display library (see, e.g., Watkins et al., Vox Sanguinis 78:72-79; U.S. Pat. Nos. 5,223,409 and 5,514,548; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734; and McCafferty et al., 1990, Nature 348:552-554, each of which is incorporated herein by reference in its entirety). The nucleic acids encoding the antibody fragment or fragments selected from the phage display library is then obtained for construction of expression vectors. The antibody fragment or fragments can then be produced in a suitable host system, such as a bacterial, yeast, or mammalian host system (see, e.g., Plückthun et al., Immunotechnology 3:83-105; Adair, Immunological Reviews 130:5-40; Cabilly et al, U.S. Pat. No. 4,816,567; and Carter, U.S. Pat. No. 5,648,237, each of which is incorporated herein by reference in its entirety).

In still another embodiment, techniques described for the production of single chain antibodies (U.S. Pat. No. 4,946,778; Bird, 1988, Science 242:423-426; Huston et al., 1988, Proc. Natl. Acad. Sci. USA 85:5879-5883; Ward et al., 1989, Nature 334:544-546; and Maynard et al., Nature Biotechnology 20:597-601, each of which is incorporated herein by reference in its entirety) can be adapted to produce single chain antibodies against the antigenic molecule. Single chain antibodies are formed by linking the heavy and light chain fragments of the Fv region via an amino acid bridge, resulting in a single chain polypeptide. Single chain antibodies can also contain, in addition to the Fv region, a constant domain of immunoglobulin.

In a specific embodiment, the invention provides a method and compositions for detecting Anthrax infection. The method comprises detecting using one or more probes that bind the protective antigen (PA) protein of Bacillus anthracis (Anthrax), a common component of the lethal and edema toxins of Anthrax (see, e.g., Little et al., 1991, Biochem Biophys Res Commun.180:531-7; Little et al., 1988, Infect Immun. 56:1807-13). In another embodiment, invention provides a method and compositions for detecting Anthrax infection using one or more probes that bind the Anthrax lethal factor (LF) and/or edema factor (EF).

### 5.3.4. Preparation of Labeled Probes

In one embodiment, quantum dots are conjugated to oligonucleotides by a method that provides strong linkage and minimizes non-specific binding of the dots themselves. In a preferred embodiment, the method as described by Pathak, et al.⁶⁷ is used to prepare quantum dots labeled polynucleotide probes. In this approach, the shell of the quantum dot is coated with dithiothreitol (DTT), a thiol compound that also contains hydroxyl groups. After the coating process, the hydroxyls are activated by treatment with 1,1′-carbonyl diimidazole to form carbamate groups. The activated groups are then coupled to 5′ or 3′ amino-oligonucleotides to form carbamate linkages.

In another embodiment, antibodies are covalently conjugated to the surfaces of quantum dots. Surfaces that have amine groups on the surface are available from the Quantum Dot Corporation, which also provides a protocol for linking the amino groups to reduced thiols on antibodies. According to the company's protocol, the surface amines are first converted to thiol-reactive maleimide groups using the hetero-bifunctional crosslinker 4-(maleimidomethyl)-1-cyclohexanecarboxylic acid N-hydroxysuccinimide ester (SMCC). Following a 60 min reaction, the excess crosslinker is removed from the activated quantum dots by gel filtration chromatography.

The other component of the conjugation reaction is the generation of free thiol groups on the antibody by reduction with DTT. Following the generation of the free thiol groups, the excess reducing reagent is also removed by gel filtration chromatography.

The maleimide-activated quantum dots are subsequently mixed with the thiol-containing antibody. Following the conjugation reaction, quenching of the excess maleimide groups is accomplished with dilute beta-mercaptoethanol. The final step of the process is the removal of any remaining free, unconjugated antibody molecules from the quantum dot conjugate. This is achieved by size-exclusion chromatography over a small column filled with Superdex® 200.

In another embodiment, QD embedded bead labels are prepared according to Han et al. (Han et al., 2001, Nature Biotechnology 19:631-635). Polystyrene beads are synthesized by using emulsion polymerization of styrene (98% vol/vol), divinylbenzene (1% vol/vol), and acrylic acid (1% vol/vol) at 70° C. Incorporation of QDs is achieved by swelling the beads in a solvent mixture containing 5% (vol/vol) chloroform and 95% (vol/vol) propanol or butanol, and by adding a controlled amount of ZnS-capped CdSe QDs to the mixture. For single-color coding with 10 intensity levels, the ratios of QDs to beads can be in the range of 640 to 50,000. For multicolor coding, the amounts of QDs can be adjusted experimentally to compensate for the different optical properties of different-colored dots. The embedding process is complete within <30 min at room temperature. Polymer beads embedded with luminescent QDs in the size range of 0.1-5.0 μm are prepared. The bead size can be controlled by changing the amount of a stabilizer (polyvinylpyrrolidone, MW=40,000) used in the synthesis. Before DNA conjugation, the encoded beads are protected by using 3-mercaptopropyl trimetroxysilane, which polymerized inside the pores upon addition of a trace amount of water. The beads are covalently attached to streptavidin molecules via the carboxylic acid groups on the bead surface. Nonspecific sites on the bead surface are blocked by using BSA (0.5 mg/ml) in PBS buffer (pH 7.4). Biotinylated oligo probes are linked to the beads via the attached streptavidin.

In another embodiment, nanoparticles embedded with dye molecules are synthesized by using a microemulsion method according to Lian et al. (Lian et al., 2004, Analytical Biochemistry 334:135-144, which is incorporated herein by reference in its entirety). To conjugate with biomolecules, the following surface modifcations can be performed on the nanoparticles: (i) silanization with the addition of 1 mM acetic acid and 1% DETA while stirring for 30 min; (ii) carboxyl modification by adding 10% succinic anhydride in dimethylformamide under nitrogen purge and stirring for at least 6 h; (iii) 1-ethyl-3-(3-dimethylaminopropyl)carbodiimide hydrochloride and NHS chemistry by adding 1% each in 0.1M 4-morpholineethanesulfonic acid buffer (pH 5.6) for 15-30 min; and (iv) the newly formed NHS-functionalized nanoparticles mixed with monoclonal antibody or avidin or streptavidin at various ratios for 2-4 h at room temperature. Remaining free NHS esters were quenched by adding hydroxylamine to 50 mM, Tris-HCl, pH 7.5, to 0.5M, and BSA to 1%.

### 5.4. Apparatuses and Computer Systems

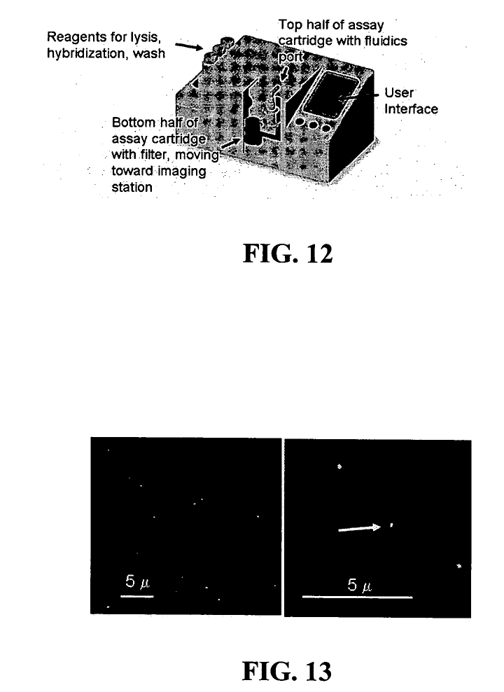

The invention provides a system that accomplishes the process diagrammed in FIG. 1. The system comprises (a) means for capturing pathogenic agents and/or cellular constituents therefrom from said sample on a surface; (b) means for labeling the surface with a probe composition comprising for each of the one or more pathogenic agents a set of one or more probes that specifically bind the pathogenic agent and/or cellular constituents therefrom under conditions that specific binding occurs, wherein each of the one or more probes in the composition has a concentration of above a given concentration threshold; (c) means for washing the surface with a wash composition to remove non-specifically bound probes, wherein the wash composition wash composition dissociates probes that bind with a binding constant less than a given non-specific binding threshold; (d) means for obtaining one or more images of the surface with a spatial resolution higher than a given resolution threshold; and (e) means for determining each of the one or more of said pathogenic agents as present in the sample if sets of one or more probes that bind the pathogenic agents is detected in the images. Preferably, the concentration threshold is at least 1 nM, 2 nM, 5 nM, 10 nM, 20 nM, 50 nM, or 100 nM or a concentration such that specific binding of the probe to at least 10%, 20%, 30%, 50%, 70%, or 90% of its target recognition sites occurs within about 1, 2, 5, 10, or 15 minutes. In a preferred embodiment, the non-specific binding threshold is fraction of the specific binding threshold. In one embodiment, the non-specific binding threshold is about 5%, 1%, 0.1%, 0.01% or 0.001% of the specific binding threshold. In another embodiment, the non-specific binding threshold is selected such that dissociation of at least a given percentage of the non-specifically bound probes occurs within a given wash time period. In one embodiment, the non-specific binding threshold is selected such that dissociation of at least half of the non-specifically bound probes occurs within about 15, 10, 5, or 1 minute, or about 30 or 10 seconds. An exemplary assay cartridge and the device platform are shown in FIGS. 11 and 12.

The device design consists of disposables and an instrument. The disposables consist of three components: a collection device, the assay cartridge, and the reagent cartridge. The first disposable is a collection device that allows the user to obtain a sample from a patient, such as nasal or throat swab, blood or other bodily fluids. In the case of blood collection it consists of a needle and a syringe. A throat swab would be aliquoted into a small-volume preloaded syringe with blunt orifice. To separate blood cells and other human cells from bacterial and viral targets, the syringe will be emptied through a 5 μ filter. The syringe has a Luer lock fitting that connects to the assay cartridge to allow the filtrate to transfer.

The assay cartridge is designed to accomplish efficient labeling of target molecules and their capture onto a surface that can be microscopically imaged. After the filtrate transfers to the assay cartridge, it combines with lysis reagents that are provided from a disposable cartridge mounted on the instrument. The mixing process is facilitated by forcing the liquid through a piston head with many pores to cause turbulent flow. To lyse spore samples, sonic energy is conveyed to the liquid by a diaphragm in the cartridge that is actuated by piezoelectric transducers that reside in the instrument. During lysis, heat is provided to accelerate lysis and denature double-stranded nucleic acids.

After lysis, hybridization reagents, including fluorescently labeled oligonucleotides are pumped from a hybridization reagent cartridge mounted in the instrument on a manifold, into the cartridge and mixed using the perforated piston head in the cartridge. The same mixing process provides rapid hybridization of the probes to the targets.

Another piston in the cartridge pushes the liquid through an ultrafilter whose molecular weight cutoff is chosen to allow capture of the target and the hybridized probes and passage of unhybridized probes. After the hybridization solution is forced through the membrane, a wash step follows. A solution provided from a wash cartridge on the instrument carries the remaining unhybridized probes through the ultrafilter. Liquid that passes through the ultrafilter is stored in the cartridge as waste below the ultrafilter. After capture of the hybridization complexes on the ultrafilter, it is covered with a coverslip and microscopically imaged. The geometry of this last step will be arranged to minimize contamination of the platform by the sample.

The instrument (FIG. 12) contains pumps to drive the fluid transfers, a sonication subsystem, an imaging subsystem, electronic subsystem, software operating system, and user interface. The fluidic subsystem includes the manifold, pumps, tubing and interfaces with the three cartridges that provide the lysis, hybridization and wash reagents. The manifold controls fluid flow and provides heating. The imaging subsystem includes the mechanism that transports the part of the cartridge that contains the ultrafilter to the optical hardware. The sonication subsystem includes piezo-electric transducers that interface with the cartridge. The optical hardware includes a laser or LED for excitation, a band pass filter to prevent stray light from reaching the sample, filters in a filter wheel to permit resolution of at least two colors of emitted light and a CCD camera. The electronic subsystem contains a central processor and supporting hardware, such as ROM and mass-storage memory. The operating system that directs the operations of the instrument and the software for the user interface reside in the electronic subsystem. The user interface hardware includes a touch screen and supporting memory.

The methods of the present invention can preferably be implemented using a computer system. An exemplary computer system suitable from implementing the methods of can comprise a processor element interconnected with a main memory. For example, the computer system can be an Intel Pentium IV®-based processor of 3.6 GHZ or greater clock rate and with 2 GB or more of main memory. The external components can include a mass storage. This mass storage can be one or more hard disks that are typically packaged together with the processor and memory. Such hard disks are typically of 10 GB or greater storage capacity and more preferably have at least 100 GB of storage capacity. The computer system of the invention can further comprise other mass storage units including, for example, one or more floppy drives, one more CD drives, one or more DVD drives, one or more DAT drives, or one or more flash drives.

Other external components typically include a user interface device, which is most typically a monitor and a keyboard together with a graphical input device such as a “mouse.” The computer system is also typically linked to a network link which can be, e.g., part of a local area network (“LAN”) to other, local computer systems and/or part of a wide area network (“WAN”), such as the Internet, that is connected to other, remote computer systems.

One or more software components are loaded into memory during operation of such a computer system. The software components comprise both software components that are standard in the art and components that are special to the present invention. These software components are typically stored on mass storage such as the hard drive, but can be stored on other computer readable media as well including, for example, one or more floppy disks, one or more CD-ROMs, one or more DVDs, one or more DATs, or one or more flash drives. Software components include an operating system which is responsible for managing the computer system and its network interconnections. The operating system can be, for example, of the Microsoft Windows™ family such as Windows XP. Alternatively, the operating software can be a Macintosh operating system, a UNIX operating system or the LINUX operating system. Software components may also include common languages and functions that are preferably present in the system to assist programs implementing methods specific to the present invention. Languages that can be used to program the analytic methods of the invention include, for example, C and C++, FORTRAN, PERL, HTML, JAVA, and any of the UNIX or LINUX shell command languages such as C shell script language. The methods of the invention can also be programmed or modeled in software packages that allow symbolic entry of equations and high-level specification of processing, including specific algorithms to be used, thereby freeing a user of the need to procedurally program individual equations and algorithms. Such packages include, e.g., Matlab from Mathworks (Natick, Mass.), Mathematica from Wolfram Research (Champaign, Ill.) or S-Plus from MathSoft (Seattle, Wash.). Software components can also include programs for controlling the apparatus, e.g., microscope, sample preparation, etc.

In addition to the exemplary program structures and computer systems described herein, other, alternative program structures and computer systems will be readily apparent to the skilled artisan. Such alternative systems, which do not depart from the above described computer system and programs structures either in spirit or in scope, are therefore intended to be comprehended within the accompanying claims.

### 5.5. Kits

The invention provides kits comprising in one or more containers a probe composition comprising for each of one or more pathogenic agents a set of one or more probes each specifically binding to a recognition site of said pathogenic agent and threshold value data on an accessible medium comprising colocalization threshold values for each of said one or more pathogenic agents, wherein said colocalization threshold values for each said pathogenic agent correspond to a degree of colocalization of said two or more probes in said set which indicates the presence or absence of said pathogenic agent. In one embodiment, each of set of different probes comprises 3 different probes. In one embodiment, each different probe is labeled with a different label such that the probes can be distinguishably detected.

The kit can also comprise one or more type-specific labels, e.g., DAPI.

In one embodiment, the kit comprises probe sets for 5, 10, 50, or 100 different pathogenic agents.

In one embodiment, the kit also comprises in a separate container a wash composition.

In one embodiment, the kit also comprises a filter for capturing pathogenic agents and/or cellular constituents therefrom.

In one embodiment, a set of probes for each pathogenic agent is contained in a separate container and the kit further comprises reagents for constructing a custom probe composition using a portion or all of the sets of probes.

### 6. EXAMPLES

The following examples are presented by way of illustration of the present invention, and are not intended to limit the present invention in any way.

### 6.1. Methods and Apparatuses

FIG. 1 illustrates a method of detection involving hybridization of the labeled probes to the target DNA occurs in solution. Alternatively, intact virions and bacteria can be captured on the filter, partially lysed and then labeled either with antibodies to surface proteins, or with DNA probes.

The invention provides a system that accomplishes the process diagrammed in FIG. 1. An exemplary assay cartridge and the device platform are shown in FIGS. 11 and 12.

The device design consists of disposables and an instrument. The disposables consist of three components: a collection device, the assay cartridge, and the reagent cartridge. The first disposable is a collection device that allows the user to obtain a sample from a patient, such as nasal or throat swab, blood or other bodily fluids. In the case of blood collection it consists of a needle and a syringe. A throat swab would be aliquoted into a small-volume preloaded syringe with blunt orifice. To separate blood cells and other human cells from bacterial and viral targets, the syringe will be emptied through a 5 μ filter. The syringe has a Luer lock fitting that connects to the assay cartridge to allow the filtrate to transfer.

The assay cartridge is designed to accomplish efficient labeling of target molecules and their capture onto a surface that can be microscopically imaged. After the filtrate transfers to the assay cartridge, it combines with lysis reagents that are provided from a disposable cartridge mounted on the instrument. The mixing process is facilitated by forcing the liquid through a piston head with many pores to cause turbulent flow. To lyse spore samples, sonic energy is conveyed to the liquid by a diaphragm in the cartridge that is actuated by piezoelectric transducers that reside in the instrument. During lysis, heat is provided to accelerate lysis and denature double-stranded nucleic acids.

After lysis, hybridization reagents, including fluorescently labeled oligonucleotides are pumped from a hybridization reagent cartridge mounted in the instrument on a manifold, into the cartridge and mixed using the perforated piston head in the cartridge. The same mixing process provides rapid hybridization of the probes to the targets.

Another piston in the cartridge pushes the liquid through an ultrafilter whose molecular weight cutoff is chosen to allow capture of the target and the hybridized probes and passage of unhybridized probes. After the hybridization solution is forced through the membrane, a wash step follows. A solution provided from a wash cartridge on the instrument carries the remaining unhybridized probes through the ultrafilter. Liquid that passes through the ultrafilter is stored in the cartridge as waste below the ultrafilter. After capture of the hybridization complexes on the ultrafilter, it is covered with a coverslip and microscopically imaged. The geometry of this last step will be arranged to minimize contamination of the platform by the sample.

The instrument (FIG. 12) contains pumps to drive the fluid transfers, a sonication subsystem, an imaging subsystem, electronic subsystem, software operating system, and user interface. The fluidic subsystem includes the manifold, pumps, tubing and interfaces with the three cartridges that provide the lysis, hybridization and wash reagents. The manifold controls fluid flow and provides heating. The imaging subsystem includes the mechanism that transports the part of the cartridge that contains the ultrafilter to the optical hardware. The sonication subsystem includes piezo-electric transducers that interface with the cartridge. The optical hardware includes a laser or LED for excitation, a band pass filter to prevent stray light from reaching the sample, filters in a filter wheel to permit resolution of at least two colors of emitted light and a CCD camera. The electronic subsystem contains a central processor and supporting hardware, such as ROM and mass-storage memory. The operating system that directs the operations of the instrument and the software for the user interface reside in the electronic subsystem. The user interface hardware includes a touch screen and supporting memory.

### 6.2. Nucleic Acids Detection

Nucleic acids can be detected using polynucleotide probes. In order to accomplish fast detection without DNA amplification, labeling is accompolished a regime of binding kinetics different from that used in most molecular assays. Instead of allowing a low concentration of ligands to slowly find their correct binding sites, as in a ˜1 hour ELISA test or overnight microarray hybridization, a high ligand concentration is used to speed up the creation of duplexes. However, this results in a large amount of non-specific binding which must then be removed by a stringent denaturing. The resulting kinetics (Lauffenburger, D. A., and Linderman, J. J., 1993, Receptors: models for binding, trafficking, and signaling, Oxford University Press, New York) were simulated and are illustrated in FIG. 2 for a set of particular parameter choices. Some general features of the association and dissociation reactions are clear. For large ligand concentrations the approach to equilibrium during association is very fast, and above a certain ligand concentration signal saturates. During wash, although signal is lost, there is a rapid increase in the ratio of signal to clutter.

In DNA-based detection, it is not necessary to retain the intact genomic DNA for detection. As shown at the lower left of FIG. 1, DNA fragments can be detected. Color coincidence detection can be used on individual fragments. The probes can be selected to by complementary to sequences within a few kilobases. Individual DNA fragments can be detected readily when tagged with superbright labels such as quantum dots. This is shown in FIG. 5, where ˜kilobase DNA fragments were each tagged with one quantum dot using biotin-streptavidin binding. Exposures of less than one second are sufficient to provide signals well above the background image noise level, using the Leica DM6000B imaging system.

This single-fragment detection capability produces very high detection efficiency in the sense that most labeled fragments are seen. Detection is limited in theory only by the statistics of the number of target fragments present in the sample. It also enables color coincidence detection approach, in which two or more independent recognition sites separated by less than the DNA fragment size (a few kilobases or less) will be assigned probes with different colors. Detection of a specific target type will be declared only when both colors are present in an image pixel. Colocalization detection of two or more differently labeled DNA hybridization probes was done in a flow cell configuration (Castro et al., 1997, Anal Chem 69, 3915-20) in 1997 and was shown to provide dramatic processing gains that enabled specific detection of individual target fragments (see, e.g., Section 5.3.).

Gel electrophoresis was used to obtain and verify isolation of dot-labeled DNA from free dots (FIG. 7). This assay also is being used to monitor hybridization products in solution between Qdot-labeled probes and target DNA so that they can be related to their appearance under fluorescence microscopy. FIG. 8 shows a mix of unbound Qdot-labeled probes, 1-kb PCR products containing complementary binding sequences for the probes, and probes specifically duplexed to the 1-kb pieces. SYBR green staining of the double stranded DNA is rendered blue and shows up along a curvilinear structure which seems to be a chain of duplexes and 1-kb fragments made possible by the fact that multiple oligos are conjugated to each Qdot via its multiple streptavidin sites. A two minute hybridization time was used.

### 6.3. Proteins Detection

Protein markers, e.g., surface antigens, are detected using antibodies that bind the markers. As an illustration, FIG. 3 shows gp64 antibody to baculovirus surface protein was used to rapidly and specifically label baculovirus virions that had been captured on a 0.2 μ pore filter. In this experiment the non-specific binding of gp64 to the filter, and of the mismatched negative control antibody to the virions in the control experiment, was washed away through the filter with a stringent 10 sec wash. In this experiment 10⁵-10⁶ virions were present on the filter. For a more dilute sample, as was assumed in generating FIG. 2, total clutter signal may still exceed total specific signal after wash, as indicated in the right part of the right frame of FIG. 2. This can be circumvented by using high resolution imaging and color coincidence detection to greatly increase the effective signal to clutter ratio.

The gain derived from resolution is a familiar concept, illustrated in FIG. 4 where two E. coli cells were stained with quantum-dot labeled antibodies in a two minute incubation. Antibodies labeled with 605 nm emission dots and antibodies labeled with 705 nm emission dots were used together. The (unfiltered) solution was imaged under cover slip with our Leica DM6000B fluorescence imaging system. The individual unbound dot-labeled antibodies are clearly seen as a granular background in both color channels. Individual quantum dots also are seen bound to the cells via the antibodies. In both color channels there is a significant total brightness in the distributed background due to the unbound probes. However, the spatial resolution makes the detection of the cells obvious, and the fact that red and green labels only tend to collocate on the cells makes the detection even stronger; basing detection on yellow (coincident) pixels only, there would be essentially zero background. The actual gain from color coincident detection involves the degree of spatial correlation (lumpiness) of the background and how these lumps correlate between the color channels. This principle holds even when the target itself is smaller than a resolution cell (pixel) of the imaging system, as will be true for most viruses and individual DNA fragments. Thinking of non-target organisms as background, color coincidence enhances detection performance because the non-target organisms, even though they may be related biologically to the target organism, are much less likely to bind both of two different probes that were designed to be specific for the target organism.

In these antibody binding experiments, adequate signal for detection built up in less than one minute, and was E. coli specific (FIG. 5). As expected from FIG. 2, detectable signal accumulated faster when higher probe concentrations were used; detections were possible within ˜5 sec when using micromolar antibody titers.

### 6.4. Probe Selection

Probe sets are designed based on pathogens of interests and operational scenarios that the test is used. Exemplary choices for these probe sets are indicated in FIG. 11 and include a set for parallel detection of all Category A agents, a set for detection and detailed discrimination of B. anthracis strains and other near-neighbor organisms in that clade, and a set for detection and detailed discrimination of RNA viruses. Additional probe sets can be added. These probe reagent sets are also provided in kits for delivery.

Genome sequence information can be retrieved from several sources including NCBI, individual databases being developed under the NLAID Bioinformatics Resource Centers for Biodefense and Emerging or Re-Emerging Infectious Diseases program (NIAID. NIAID Bioinformatics Resource Centers for Biodefense and Emerging or Re-Emerging Infectious Diseases Program, http://www.niaid.nih.gov/dmid/genomes/brc/default.htm), and individual databases. An informatics infrastructure is assembled including a database of genomic sequence representing Category A, B, and C pathogens and strain variants, probe design algorithms, and software linking the two.

Where possible, target recognition sequences for each threat organism will be chosen that are intimately related to its specific known virulence properties and mechanisms, as in the approach to the B. anthracis clade (Kim et al., 2005, FEMS Immunology and Medical Microbiology 42:301-310). In another embodiment, a detailed phylogenetic analysis of the clade surrounding each threat organism will be done to identify likely near-neighbor false positives and a biological basis for the choice of gene regions most likely to provide robust and specific detection (see, e.g., Kim et al., 2005, FEMS Immunology and Medical Microbiology 42:301-310).

In one embodiment, probe design in our approach involves choosing two or more identification sites per target sequence for oligo probe binding where these sites are separated by ˜5000 nucleotides or less to support spatial coincidence detection. At the same time, each label type can be assigned to several probes targeting different recognition sites widely separated over the genome, creating even more robust detection. In one embodiment, commercial softwares (e.g., ArrayDesigner, by Premier Biosoft International; TILIA, by Linden Biosciences) and public software (Li et al., 2001, Bioinformatics 17, 1067-76) are used for designing hybridization probes.

The probes need not be of the same length. In preferred embodiments, probes having different lengths but uniformed binding constant, e.g., constant Tm, are selected. In one embodiment, probe length is varied around 30 nucleotides to achieve roughly constant Tₘ so that an optimal trade between sensitivity and specificity can be made simultaneously for multiple probes. Tₘ can be computed based on a nearest neighbor model of solution phase oligo hybridization with quartet energy coefficients taken from published values for perfect match and mismatch quartets (SantaLucia et al., 1996, Biochemistry 35, 3555-62; SantaLucia et al., 1997, Biopolymers 44, 309-19; Sugimoto et al., 1995, Biochemistry 34, 11211-6; Sugimoto et al., 1996, Nucleic Acids Res 24, 4501-5). The steric effects of quantum dots on the hybridization can also be evaluated for refinement of the probe design rules. Probes can further be selected to avoid sequences with propensity for secondary structure, avoid low-complexity sequence, and avoid cross-hybridization to other targets. The cross-hybridization calculation is a computationally demanding but important part of the process. It can also consider the possible presence of other common infectious agents not on the NIAID Category A, B, C lists such as adenoviruses, rotoviruses, and common influenzas associated with upper respiratory and flu-like symptoms. It also will consider commensal organisms that often are carried without overt disease. Examples of these agents include (Heritage, 2003, The Human Commensal Flora, Leeds University Website) Herpesvirus simplex 1 (HSV1) associated with cold sores in the mouth mucosa, Streptococcus mutans associated with placque and tooth decay, Staphylococcus aureus often carried in the nose, Streptococcus pneumoniae, Streptococcus pyogenes and Neisseria meningitides often found in the throat. For nasal swabs, environmental background organisms need to be considered. These are potentially more diverse than those actually growing in the nasal passage, and include pollens and common airborne environmental bacteria such as Bacillus subtilis, Bacillus cereus, Bacillus thuringiensis, Burkholderia cepacia, and Ralstonia solanacearum. In particular, B. cereus and B. thuringiensis both are very close relatives of B. anthracis and will be distinguished carefully in the probe design as described above.

In operation scenarios where symptoms provide prior information, a probe composition can include probes for a panel of infectious agents that may cause the symptom. The sequence database will be augmented with the genomes of these common and commensal agents.

### 6.5. Quantitative Colocalization Determination

This example illustrates quantitative colocalization determination. E. coli cells were labeled with antibodies labeled with 605 nm “green” QD and 705 nm “red” QD using a 2-minute hybridization to Qdot-labeled antibodies of two different colors. A 256×256 pixel image region containing E. coli cells were analyzed. FIG. 14A shows the original image with intensity transform ‘gamma’ chosen to reveal background clutter associated with the individual labeled antibodies, as well as the bacterial cells. FIG. 14B shows a composite image composed of the pixel by pixel intensity product. It can be seen that signal-to-clutter ratio is significantly improved. FIG. 14C shows the intensity profile along the blue dashed line in FIG. 14B. The thick line is the product intensity, which has a much higher signal to noise ratio across the bacterium features than does each of the individual color channels.

### 6.6. Tests of Detection Performance

Tests of detection performance are carried out using surrogates as shown in Table 1.

TABLE 1
Surrogate organisms used in the tests.

| Surrogate Organism | Threat Category | Representative Threats | Genome Type |
|---|---|---|---|
| Escherichia coli K12 | Vegetative Bacteria | Yersinia pestis (plague) | DNA, circular |
| Bacillus cereus ATCC 14579 | Sporulating Bacteria | Baccillus anthracis | DNA, circular |
| Bacillus thuringiensis serovar | Sporulating Bacteria | Baccillus anthracis | DNA, circular |
| israelensis | | | |
| Autographa califomica | DNA Virus | Variola virus (smallpox) | dsDNA, circular |
| nucleopolyhedrovirus | | | |
| Enterobacterio phage MS2 | RNA Virus | Ebola virus, Marburg virus | ssRNA, linear |

These organisms are spiked into human blood, urine, and sputum samples in known concentrations to make synthetic test samples to support demonstration of fundamental performance parameters such as specificity, sensitivity, and speed. In antibody labeling tests ovalbumin is used as a surrogate for toxins such as Botulinum and Staphylococcus enterotoxin. The diversity of the synthetic samples is increased by including inactivated partial genomes of real Category A and B threats and/or synthetic DNA sequences representing the target identification regions of these threats. When synthetic target sequences are used, the complexity of a full target genome is simulated by including a comparable mass of genomic DNA from the appropriate BL1 surrogates in Table 1.

Actual tests with viable BSL-2 viral agents vaccinia and Vesicular Stomatitis Virus (VSV) are conducted. These tests provides practice in delivering a detection system off-site, as well as practice with viable viral agents.

Vaccinia and VSV are good surrogates for Category A and B viruses. The NIH list of Category A and B viral agents includes only one DNA virus, smallpox. Vaccinia is 95% identical to smallpox at the nucleotide level, and is a favored model system for basic molecular studies of poxviruses. Different strains of vaccinia virus, e.g., the Copenhagen and the WR strain, have been used for a number of studies over the last decade. Both are BSL-2 agents. The attenuated Ankara (MVA) strain also can be obtained and handled at the BSL-2 level. About 15% of the vaccinia genome is deleted in the MVA strain which also contains numerous additional mutations. The WR and Copenhagen strains on the other hand are very closely related (>98% identity). These three vaccinia virus strains therefore present a useful range of sequence diversity for the design and testing of specific probes.

A much larger number of RNA viruses (19), all single-stranded, are considered Category A or B agents. These are either non-segmented, positive-strand RNA viruses such as Dengue, West Nile, hepatitis A, and Venezuelan equine encephalitis virus, non-segmented, negative-strand RNA viruses such as filoviruses, or segmented, negative-strand RNA viruses such as lymphochoriomeningitis virus (LCM), hantaviruses, and La Crosse virus. The diversity of agents and the small size of their genomes which are <15 kb, some in segments <3 kb, pose challenges to the design of probes and appropriate choice of surrogates. In addition, single-stranded RNA genomes are far more susceptible to degradation by nucleases once released from their protective capsids. Most of these agents do not have well characterized close relatives that can be handled at the BSL-2 level. VSV (vesicular stomatitis virus) however is a very well characterized, non-segmented, negative strand RNA virus that shares many of the features of these RNA viruses. The two VSV strains are utilized, the Indiana and New Jersey serotypes, which have genome sequences that differ overall by ˜30%, but conservation varies widely depending on the particular gene or subgenic region. VSV should pose similar challenges in probe design and detection as Category A and B RNA viruses.

The fundamental performance criteria for detection tests involve Probabilities of Detection (P_{D}) and Probabilities of False Positive (P_{FA}) on clinical samples. The synthetic samples contain known quantities of surrogate threat material, including the case of zero threat as negative control. For tests involving parallel detection of many agents, the sample contains only one or a few of the threats in non-zero quantity. False positives is assessed for the threats which were probed for but not included in the sample. A typical round of testing includes ˜20 independently created samples with ˜10 threats probed for in parallel. Thus false positive statistics is obtained for 20×10=200 threat hypotheses, which provides enough statistical stability to estimate P_{FA}. Tests are run at different spike-in levels to establish the lower limit of detection that can be achieved while maintaining a useful P_{D} and P_{FA}. The robustness to interfering human genomic DNA is also tested by adding known concentrations of human DNA. These tests establishes the following probabilities of detection and of false alarms at the lower limit of detection: P_{D}>0.95 averaged over the test organisms and P_{FA}<0.01 summed over all the threat hypotheses tested and averaged over the tests.

A small number of pre-existing irreversibly anonymized clinical samples suspected or known to contain particular common respiratory pathogens such as influenza A is used for detection tests on actual infected clinical samples. These limited small-scale tests confirm that the results obtained with synthetic samples are reliable in demonstrating the performance of the methods.

### 7. REFERENCES CITED

All references cited herein are incorporated herein by reference in their entirety and for all purposes to the same extent as if each individual publication or patent or patent application was specifically and individually indicated to be incorporated by reference in its entirety for all purposes.

Many modifications and variations of the present invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

What is claimed is:

1. A method for determining whether a sample comprises a target pathogenic agent, said method comprising
(a) determining quantitatively a degree of colocalization of a plurality of different probes on a surface, wherein any one or more pathogenic agents and/or cellular constituents therefrom from said sample are fixed on said surface, by calculating a metric of colocalization between a plurality of detection channels each corresponding to one of said probes, wherein each said different probe specifically binds a different one of a plurality of recognition sites, and wherein said plurality of different recognition sites are colocalized in said target pathogenic agent or a cellular constituent of said target pathogenic agent; and
(b) determining that said sample comprises said target pathogenic agent if said degree of colocalization of said plurality of different probes on said surface is higher than a predetermined threshold.

2. The method of claim 1, wherein said step (a) is carried out by a method comprising
(i) contacting said surface with a probe composition comprising said plurality of different probes under conditions that specific binding of said probes to their respective recognition sites occurs;
(ii) detecting said plurality of different probes on said surface; and
(iii) determining said degree of colocalization.

3. A method for determining whether a sample comprises a target pathogenic agent, said method comprising
(a) contacting a surface, wherein any one or more pathogenic agents and/or cellular constituents therefrom from said sample are fixed on said surface, with a probe composition comprising a plurality of different probes under conditions such that specific binding of said probes to their respective recognition sites occurs, wherein each said different probe specifically binds a different one of a plurality of recognition sites, wherein said plurality of different recognition sites are colocalized in said target pathogenic agent or said cellular constituent;
(b) detecting said plurality of different probes on said surface;
(c) determining quantitatively a degree of colocalization of said plurality of different probes on said surface by calculating a metric of colocalization between a plurality of detection channels each corresponding to one of said probes; and
(d) determining that said sample comprises said target pathogenic agent if said degree of colocalization of said plurality of different probes on said surface is higher than a predetermined threshold.

4. The method of claim 3, wherein said plurality of probes comprises 3 different probes.

5. The method of claim 3, wherein said plurality of probes comprises 5 different probes.

6. The method of claim 3, wherein two of said probes are each labeled with a fluorescence label, the fluorescence labels having one of a different emission wavelength and a different excitation wavelength from one another.

7. The method of claim 3, wherein said plurality of different probes is labeled with a predetermined number of each of a plurality of different fluorescence labels.

8. The method of claim 6, wherein two of said probes are each labeled with a fluorescence label, the fluorescence labels having both a different emission wavelength and a different excitation wavelength from one another.

9. The method of claim 3, wherein said plurality of recognition sites comprises a plurality of DNA sequences of said target pathogenic agent, wherein said DNA sequences are located in an approximately 2 kb or less region of DNA sequence of said target pathogenic agent.

10. The method of claim 3, wherein said plurality of recognition sites comprises a plurality of DNA sequences of said target pathogenic agent, wherein said DNA sequences are located in an approximately 1 kb or less region of DNA sequence of said target pathogenic agent.

11. The method of claim 3, wherein said probe composition further comprises a type-specific label, said method further comprising the step of detecting said type-specific label and determining colocalization of plurality of probes on image regions also labeled with said type-specific label.

12. The method of claim 11, wherein said type-specific label is DAPI.

13. The method of claim 3, wherein said plurality of recognition sites comprises a plurality of surface antigens of said target pathogenic agent.

14. The method of any one of claims 1 and 3, wherein said degree of colocalization is represented by a metric comprising an overlap coefficient of a pair of said plurality of detection channels.

15. The method of any one of claims 1 and 3, wherein said degree of colocalization is represented by a metric comprising colocalization coefficients m₁ and m₂ of a pair of said plurality of detection channels.

16. The method of any one of claims 1 and 3, wherein said degree of colocalization is represented by a metric comprising at least a Pearson correlation coefficient of a pair of said plurality of detection channels.

17. The method of any one of claims 1 and 3, wherein said target pathogenic agent further comprises a second plurality of different recognition sites that are colocalized, wherein said probe composition further comprises a second plurality of different probes each specifically binding one of said second plurality of recognition sites, wherein said method further comprises before step (d) repeating steps (b) and (c) with said second plurality of probes, and determining that said sample comprises said target pathogenic agent if a degree of colocalization of said second plurality of different probes on said surface is also higher than a second predetermined threshold.

18. The method of claim 17, wherein said plurality of recognition sites comprises a plurality of DNA sequences of said target pathogenic agent, wherein said DNA sequences are located in an approximately 1 kb or less region of DNA sequence of said target pathogenic agent, and wherein said second plurality of recognition sites comprises a plurality of surface antigens of said target pathogenic agent.

19. A method for determining whether a sample comprises a plurality of different target pathogenic agents, wherein each said target pathogenic agent comprises a plurality of different recognition sites that are colocalized, said method comprising
(a) contacting a surface, wherein any one or more pathogenic agents and/or cellular constituents therefrom from said sample are fixed on said surface, with a probe composition comprising a plurality of sets of different probes under conditions that specific binding of said probes to their respective recognition sites occurs, wherein each said set comprises a plurality of different probes each specifically binding one of said plurality of recognition sites;
(b) detecting said plurality sets of different probes on said surface;
(c) determining quantitatively for each said set a degree of colocalization of said plurality of different probes on said surface by calculating a metric of colocalization between a plurality of detection channels each corresponding to one of said probes; and
(d) determining that said sample comprises a target pathogenic agent if said degree of colocalization of the corresponding set of probes on said surface is higher than a predetermined threshold.

20. The method of claim 19, wherein said plurality of different target pathogenic agents comprises 5 different target pathogenic agents.

21. The method of claim 19, wherein said plurality of different target pathogenic agents comprises 100 different target pathogenic agents.

22. The method of claim 19, wherein each of said sets of different probes comprises 3 different probes.

23. The method of claim 22, wherein each said different probe is labeled with one of ten different labels such that each set of different probes has a unique combination of different labels.

24. The method of claim 23, wherein said ten different labels are ZnS-capped CdSe quantum dots having emission wavelengths at approximately 443, 473, 481, 500, 518, 543, 565, 587, 610, and 655 nm, respectively.

25. The method of claim 19, wherein said plurality of recognition sites comprises a plurality of DNA sequences of said target pathogenic agent, wherein said DNA sequences are located in an approximately 2 kb or less region of DNA sequence of said target pathogenic agent.

26. The method of claim 19, wherein said plurality of recognition sites comprises a plurality of DNA sequences of said target pathogenic agent, wherein said DNA sequences are located in an approximately 1 kb or less region of DNA sequence of said target pathogenic agent.

27. The method of claim 19, wherein said probe composition further comprises a type-specific label, and said method further comprising detecting said type-specific label and determining colocalization of plurality of probes on image regions also labeled with said type-specific label.

28. The method of claim 27, wherein said type-specific label is DAPI.

29. The method of claim 19, wherein said degree of colocalization is represented by a metric comprising an overlap coefficient of a pair of said plurality of detection channels.

30. The method of claim 19, wherein said degree of colocalization is represented by a metric comprising colocalization coefficients m₁ and m₂ of a pair of said plurality of detection channels.

31. The method of claim 19, wherein said degree of colocalization is represented by a metric comprising at least a Pearson correlation coefficient of a pair of said plurality of detection channels.

32. The method of any one of claims 1, 3 and 19, wherein said predetermined threshold is determined using one or more reference samples, each comprising a predetermined number of copies of each said target pathogenic agent.

33. A computer system comprising
a processor, and
a memory coupled to said processor and encoding one or more programs, wherein said one or more programs cause the processor to carry out the method of any one of claims 1, 3 and 19.

34. A computer program product for use in conjunction with a computer having a processor and a memory connected to the processor, said computer program product comprising a computer readable storage medium having a computer program mechanism encoded thereon, wherein said computer program mechanism may be loaded into the memory of said computer and cause said computer to carry out the method of any one of claims 1, 3 and 24.

35. A kit comprising (a) in one or more containers a probe composition comprising for each of one or more pathogenic agents a set of two or more probes each specifically binding to a recognition site of said pathogenic agent; and (b) threshold value data on an accessible medium comprising colocalization threshold values for each of said one or more pathogenic agents, wherein said colocalization threshold values for each said pathogenic agent correspond to a degree of colocalization of said two or more probes in said set which indicates the presence or absence of said pathogenic agent.

36. The kit of claim 35, wherein each of said sets of different probes comprises 3 different probes.

37. The kit of claim 36, wherein each said different probe is labeled with one of ten different labels such that each set of different probes has a unique combination of different labels.

38. The kit of claim 37, wherein said ten different labels are ZnS-capped CdSe quantum dots having emission wavelengths at approximately 443, 473, 481, 500, 518, 543, 565, 587, 610, and 655 nm, respectively.

39. The kit of claim 35, wherein said plurality of recognition sites comprises a plurality of DNA sequences of said target pathogenic agent, wherein said DNA sequences are located in approximately a 2 kb or less region of DNA sequence of said target pathogenic agent.

40. The kit of claim 35, wherein said plurality of recognition sites comprises a plurality of DNA sequences of said target pathogenic agent, wherein said DNA sequences are located in approximately a 1 kb or less region of DNA sequence of said target pathogenic agent.

41. The kit of claim 35, wherein said probe composition further comprises a type-specific label.

42. The kit of claim 41, wherein said type-specific label is DAPI.

43. The kit of claim 35, wherein said one or more pathogenic agents comprises 5 different pathogenic agents.

44. The kit of claim 43, wherein said set of probes for each said one or more pathogenic agents is in a separate container, and wherein said kit further comprises reagents for constructing a probe composition using at least a portion of said sets of probes.

45. The kit of any one of claims 35-43, further comprising in a separate container a wash composition.

46. The kit of claim 35, wherein said one or more pathogenic agents comprises 50 different pathogenic agents.

47. A method for determining whether a sample comprises a target nucleic acid or protein, said method comprising
(a) determining quantitatively a degree of colocalization of a plurality of different probes on a surface, wherein any one or more nucleic acids or proteins from said sample are fixed on said surface, by calculating a metric of colocalization between a plurality of detection channels each corresponding to one of said probes, wherein each said different probe specifically binds a different one of a plurality of recognition sites, and wherein said plurality of different recognition sites are colocalized in said target nucleic acid or protein; and
(b) determining that said sample comprises said target nucleic acid or protein if said degree of colocalization of said plurality of different probes on said surface is higher than a predetermined threshold.

48. The method of claim 47, wherein said step (a) is carried out by a method comprising
(i) contacting said surface with a probe composition comprising said plurality of different probes under conditions that specific binding of said probes to their respective recognition sites occurs;
(ii) detecting said plurality of different probes on said surface; and
(iii) determining said degree of colocalization.

49. A method for determining whether a sample comprises a target nucleic acid or protein, said method comprising
(a) contacting a surface, wherein any one or more nucleic acids or proteins from said sample are fixed on said surface, with a probe composition comprising a plurality of different probes under conditions such that specific binding of said probes to their respective recognition sites occurs, wherein each said different probe specifically binds a different one of a plurality of recognition sites, wherein said plurality of different recognition sites are colocalized in said target nucleic acid or protein;
(b) detecting said plurality of different probes on said surface;
(c) determining quantitatively a degree of colocalization of said plurality of different probes on said surface by calculating a metric of colocalization between a plurality of detection channels each corresponding to one of said probes; and
(d) determining that said sample comprises said target nucleic acid or protein if said degree of colocalization of said plurality of different probes on said surface is higher than a predetermined threshold.

50. A computer system comprising
a processor, and
a memory coupled to said processor and encoding one or more programs, wherein said one or more programs cause the processor to carry out the method of claim 49.

51. A computer program product for use in conjunction with a computer having a processor and a memory connected to the processor, said computer program product comprising a computer readable storage medium having a computer program mechanism encoded thereon, wherein said computer program mechanism may be loaded into the memory of said computer and cause said computer to carry out the method of claim 49.

52. The method of any one of claims 1, 3, 19, 47 and 49, wherein said sample and/or cellular constituents therefrom has not been subject to in vitro amplification of nucleic acids prior to said obtaining step.
